(19) United States
(12) Patent Application Publication
(10) Pub. No.: US 2024/0426819 A1
(43) Pub. Date: Dec. 26, 2024

(54) Lateral Flow Device for Allograft Rejection

(71) Applicant: NephroSant, Inc., San Mateo, CA (US)

(72) Inventors: Chih-Hung LAI, San Mateo, CA (US); Robert ZAWADA, San Mateo, CA (US); Jeff STOLL, San Mateo, CA (US); Francsico Dias LOURENCO, San Mateo, CA (US)

(21) Appl. No.: 18/755,023

(22) Filed: Jun. 26, 2024

Related U.S. Application Data
(60) Provisional application No. 63/523,273, filed on Jun. 26, 2023.

(51) Int. Cl. G01N 33/543; G01N 21/78; G01N 33/53; G01N 33/68

(52) U.S. Cl. CPC G01N 33/54388; G01N 21/78; G01N 33/5308; G01N 33/6893; G01N 2333/76; G01N 2800/245

(57) ABSTRACT

The disclosure provides lateral flow devices for detecting the presence or absence of methylated cIDNA and one or more protein(s) in a biological sample, such as urine or blood.

### CROSS-REFERENCE

This application is a Non Provisional patent application which claims the benefit of priority to U.S. Provisional Patent Application No. 63/523,273, filed Jun. 26, 2023, the entirety of which is incorporated herein by reference.

### BACKGROUND

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Lateral flow assays (LFAs) provide some of the most attractive point-of-care instruments for broad applications with simple, rapid, user-friendly, and cost-effective characteristics. However, these technologies suffer from low sensitivity, the poor limit of detection, and just qualitative or semi-quantitative results that restrict their practical applications. Extensive research has been reported in this area involving sensitivity enhancement, multiplex analysis, the implementation for broad analytes, and development of novel electronic readers for quantitative analysis.

### SUMMARY

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

In some aspects the disclosure provide a lateral flow device for detecting the presence or absence of methylated cIDNA and one or more protein(s) in a biological sample comprising: a sample application area; a capture area comprising a membrane having at least a first detectable moiety attached thereto whereby the first detectable moiety specifically binds to a methylated cell free nucleic acid (m-cIDNA) to form a detectable complex; a second detectable moiety attached thereto whereby the second detectable moiety specifically reacts with one or more protein(s); and a flow path from the sample application area to the capture area.

In other cases, the disclosure provides a lateral flow device for detecting the presence or absence of methylated cIDNA and one or more protein(s), said device comprising a test strip having a first and second end and comprising: (a) a sample receiving zone at or adjacent said first end of said test strip for receiving an aliquot of a bodily fluid sample; (b) a first capture zone in lateral flow contact with said sample receiving zone, said capture zone comprising at least a first detectable moiety attached thereto and coupled to a first binding partner which specifically binds to a methylated cell free nucleic acid to form a detectable complex; (c) a second capture zone in lateral flow contact with said first labeling zone, said capture zone comprising a dye attached thereto which specifically binds to one or more protein(s); and (c) optionally a third capture zone in lateral flow contact with said second capture zone, said capture zone comprising a control detectable moiety attached thereto which binds to a control; and (d) optionally an absorbent zone positioned at or adjacent said second end of said test strip in lateral flow contact with capture zones.

The threshold of detection for the first detectable moiety can be adjusted depending on the application. In some instances a combined threshold of detection for forming a detectable m-cIDNA complex of at least 25 ng/ml and for forming a detectable m-cIDNA complex of at least 25 ng/mL identifies an allograft rejection with at least 85% sensitivity. In preferred cases, the allograft rejection is a kidney allograft. In other cases, a threshold can be used to detect kidney lllJury.

The threshold of detection for the first detectable moiety can be adjusted depending on the application. In some instances a combined threshold of detection for forming a detectable m-CIDNA complex of at least 25 ng/ml and for forming a detectable m-cIDNA complex of at least 25 ng/mL identifies an allograft rejection with at least 65% specificity. In preferred cases, the allograft rejection is a kidney allograft. In other cases, a threshold can be used to detect kidney lllJury.

In some instances, either the second detectable moiety or the dye that specifically reacts with one or more protein(s) substantially reacts with albumin. Total protein (TO) can also be detected and a variety of dyes and binding agents can be used for protein detection (e.g., anti-albumin antibodies or protein color dies).

In many instances, the first detectable moiety is an antibody, such as an antibody for detection methylated cell-free DNA (abbreviated m-cIDNA or methyl-cIDNA). The antibody can be a pan methyl antibody for detection of methylation in nucleic acids.

In many instances, the second detectable moiety is a color changing dye, such as a color changing dye that preferably binds albumin.

In many instances, the capture area of a lateral flow device of the disclosure further comprises a control detectable moiety. In many instances, the capture area of a lateral flow device of the disclosure extends the length of the test strip.

In many instances, the control detectable moiety is a species specific IgG, such as a human IgG or a dog IgG.

In some aspects, the disclosure also provides a method for using a lateral flow device of the disclosure in detecting a kidney allograft rejection, the method comprising providing a biological sample of a subject in the capture area and determining if the sample meets a designated threshold for detection of a m-cIDNA marker and one or more protein(s).

In some aspects, the disclosure provides a kit comprising a lateral flow device of the disclosure and instructions for use therefore.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other features and advantages of the present invention will be more fully understood from the following detailed description of illustrative embodiments taken in conjunction with the accompanying drawings in which:

FIG. 1 depicts an exemplary lateral flow device for the detection of Kidney Transplant Rejection.

FIG. 2 depicts an exemplary components of a lateral flow device for the detection of Kidney Transplant Rejection.

FIG. 3 depicts an exemplary positive, negative, and invalid read-outs of lateral flow devices for the detection of Kidney Transplant Rejection.

FIG. 4 depicts an exemplary color read-outs for detection of albumin/protein biomarkers on an exemplary lateral flow device for the detection of Kidney Transplant Rejection.

FIG. 5 is a plot of the m-cIDNA values vs. the Q-Score for each sample.

FIG. 6 is a log-linear plot of m-cIDNA values (blue) and total protein values (orange) vs. the Q-Score for each sample.

FIG. 7 is a plot of OR (m-cIDNA 2:25, total protein 2:25) vs. Q-Score for each sample.

It should be understood that the drawings are not necessarily to scale, and that like reference numbers refer to like features.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Following the initial technical challenge of implanting an organ m a transplantation procedure, maintaining the organ against a vast array of pathologies for years to come, remains a challenge for all clinicians working in transplantation. Drug toxicity, opportunistic infection, primary disease recurrence, and the constant battle against organ rejection are all differentials that are considered when graft dysfunction is observed, promoting a lifetime of laborious surveillance.

After organ transplantation, monitoring patients for evidence of rejection is essential for mitigating graft loss. Diagnosis of rejection of solid organ transplants traditionally requires needle biopsy and histological assessment, which in some healthcare models can be costly, logistically challenging and carries the risk of procedure-related complications with associated morbidity. There remains a critical unmet need for an easy to use, non-invasive product, that can provide more than a mere inference of potential allograft injuries, but that is also sensitive enough and accurate enough to eliminate the need for a needle biopsy or histological assessment.

There are multiple challenges in requiring the recipient of an allograft to frequently be submitted to invasive procedures, many of which are exacerbated during pandemic and social restrictions. First, a sample should be obtained in a non-intrusive, or minimally intrusive manner.

Second, the sample must be a source of informative biomarkers for monitoring transplant health and injuries. Third, there is a need for detecting the biomarkers in a reliable, reproducible, and robust manner. Lastly, there is a need for an analysis of the data, which can require transforming data obtained by quantitative detection of biomarkers to create a composite score for a condition being studied, e.g. acute rejection (AR), allograft hypoxia, etc. To overcome the deficiencies of the current standard-of-care for transplant monitoring, a clinical test must be able to extract enough information from samples to provide a precise prognosis of an allograft.

Urine, a biofluid produced by the kidneys, can be a source of informative biomarkers for allografts. The kidneys, collectively known as the renal system, perform the essential function of removing waste products from the blood and regulating the water fluid levels. They are essential in the urinary system, but also serve homeostatic functions such as the regulation of electrolytes, maintenance of acid-base balance, and regulation of blood pressure. They serve the body as a natural filter of the blood and remove waste products which are diverted to the urinary bladder. In producing urine, the kidneys excrete waste products such as urea and ammonium, and they are also responsible for the reabsorption of water, glucose, and amino acids. If the right biomarker, or combination of biomarkers, is (are) identified, a urine sample can provide a suitable, non-invasive source of material for the evaluation of a solid allograft status. Urine can contain sufficient biomarkers to inform the status of kidney allografts with high sensitivity and accuracy, and it may be able to inform the status of other allografts as well. However, to date, there are no commercially available rapid tests that relate to the detection of kidney injury. It remains necessary to identify point of care strategies that can help identify kidney injury, particularly kidney injury caused by allograft rejection.

Traditionally, urine strips have been used to identify the levels of protein in the urine, particularly as a point-of-care tests that has been used for many years in the physician's office and hospital clinic as first-line measures of the presence of proteinuria, in order to detect chronic kidney disease (CKD). Such tests, however, have never been found to be sufficiently accurate for the detection of either CKD or another form of kidney injury, such as allograft rejection. For instance, a semi-quantitative urine strip test, Multistix PRO® IOLS (Siemens Medical Solutions, Tarrytown, USA), incorporated dual protein reagent pads-‘Protein-Low’ and ‘Protein-High’-alongside the usual, more traditional, tests comprising up to IO chemical pads that can serve for the analysis of different parameters (e.g., proteins, pH, erythrocytes, leukocytes, nitrites, glucose, ketones). The chemical pads change color after being immersed in the sample, and the results can be interpreted by comparison of the pad color with the colors presented in the dipstick analysis guide. The Protein-Low pad was more specific for albumin and augmented the traditional protein pad, while still being reported as protein. The strip test was also unique in that it included a test pad for creatinine allowing for the semi-quantitative estimation of the P: C ratio, which demonstrates effective performance in detecting proteinuria, especially when the strips are read using an automated strip reader as opposed to interpreted by comparison. See, e.g., “Use of a first-line urine protein-to-creatinine ratio strip test on random urines to rule out proteinuria in patients with chronic kidney disease,” Mark Guy, Ronald Newall, Joanna Borzomato, Philip A Kalra, Christopher Price, Nephrology Dialysis Transplantation, Volume 24, Issue 4, April 2009, Pages 1189-1193. Analysis of protein on its own, even when considered in regards to its ration to creatinine, was insufficient for the detection of kidney injury.

The instant disclosure investigated a performance of a urinary composite score of five to six biomarkers—an inflammation biomarker (e.g., CXCL-10, also known as IP-10); an apoptosis biomarker (e.g., clusterin); a cIDNA biomarkers; a DNA methylation biomarker; a creatinine biomarker; and total protein in detecting kidney injury, to identify biomarkers for use with a lateral flow device for detecting kidney injury on a urine sample. The strips are expected to elicit specificity predictive values in the range of 70% and above and specificity predictive values in the range of 85% using all the random urines. Receiver-operator characteristic curve analysis of the performance of the individual biomarkers also demonstrated good performance with all samples.

The strip test allows a subject to rule out significant kidney allograft rejection on a random urine sample at home, in a doctors office, i.e., in any location, obviating the need for specially collected samples, and with the added benefit of reducing the need for a lengthy and costly quantitative laboratory analysis of the lateral flow test.

Lateral Flow Devices for Detecting Protein and m-cIDNA in Urine

Most lateral flow tests reported to date relate to immunodiagnostics and are solely based on the specific interaction between antigens and antibodies. Lateral flow assays based on antibody directly immobilized in the capture zone, however, suffer from various drawbacks, including denaturation, aggregation, precipitation, variability and nonspecific and suboptimal binding. The present disclosure addresses these challenges and provides lateral flow device(s) with strategies for capturing methylated cell-free nucleic acids (methyl-cIDNA or m-cIDNA) and one or more protein(s) from a urine sample.

In some aspects, the disclosure provides a lateral flow device for detecting the presence or absence of methylated cIDNA and one or more protein(s) in a biological sample comprising: a sample application area; a capture area comprising a membrane having at least a first detectable moiety attached thereto whereby the first detectable moiety specifically binds to a methylated cell free nucleic acid (m-cIDNA) to form a detectable complex; a second detectable moiety attached thereto whereby the second detectable moiety specifically reacts with one or more protein(s); and a flow path from the sample application area to the capture area.

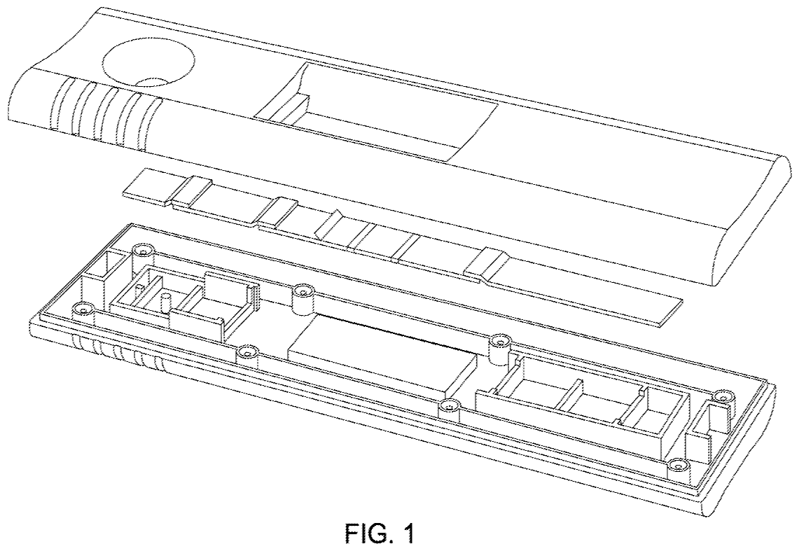

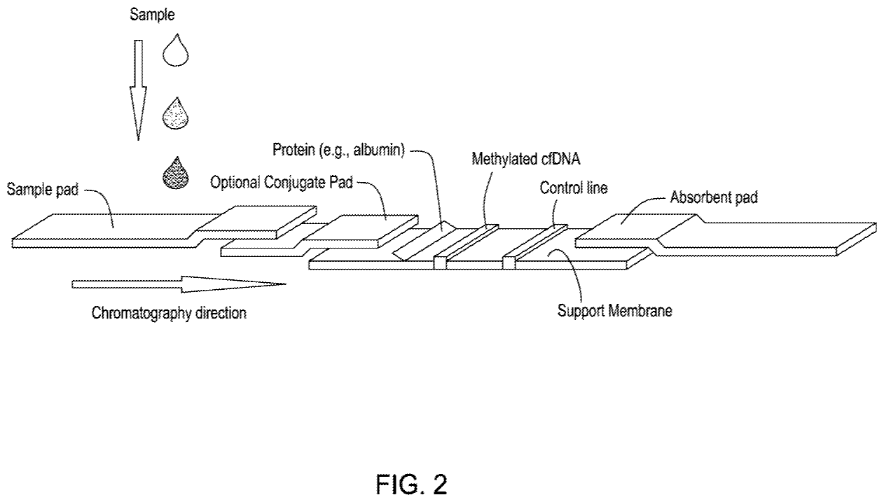

In some aspects, the disclosure provides a lateral flow device for detecting the presence or absence of methylated cIDNA and one or more protein(s), said device comprising a test strip having a first and second end and comprising: a sample receiving zone at or adjacent said first end of said test strip for receiving an aliquot of a bodily fluid sample; a first capture zone in lateral flow contact with said sample receiving zone, said capture zone comprising at least a first detectable moiety attached thereto and coupled to a first binding partner which specifically binds to a methylated cell free nucleic acid to form a detectable complex; a second capture zone in lateral flow contact with said first labeling zone, said capture zone comprising a dye attached thereto which specifically binds to one or more protein(s); and optionally a third capture zone in lateral flow contact with said second capture zone, said capture zone comprising a control detectable moiety attached thereto which binds to a control; and optionally an absorbent zone positioned at or adjacent said second end of said test strip in lateral flow contact with capture zones. FIG. 1 and FIG. 2 depicts an exemplary lateral flow device for the detection of Kidney Transplant Rejection, as well as exemplary components of a lateral flow device for the detection of Kidney Transplant Rejection, respectively.

### Detection of One or More Protein(s)

Total Protein is a well-known marker of glomerular disease. Many studies have shown that increased protein in the urine, e.g., proteinuria and albuminuria, is commonly found in kidney transplant recipients and has a strong association with acute kidney injury (AKI) and is predictive of end stage renal disease (ESRD). Albuminuria has been shown to be a reliable marker for kidney transplant rejection, occurring in up to 45% of rejection patients. Proteinuria after kidney transplant has been correlated with decreased allograft and patient survival; proteinuria is a late marker of kidney damage.

Normal daily protein excretion should not exceed 150 mg/24 hours or 10 mg/100 mL. Proteinuria is defined by the production of >1 50 mg/day with nephrotic syndrome producing >3.5 g/day. Much of this protein is the type called albumin. But many other types of protein may be found in urine. Dipstick urinalysis generally detects one or more protein(s) with bromphenol blue indicator dye and is most sensitive to albumin and less sensitive to Bence-Jones protein and globulins. Trace positive results are equivalent to 10 mg/100 ml or about 150 mg/24 hours (the upper limit of normal).

However, urine dipstick test results may vary depending on a subject's hydration status. Thus, even trace proteinuria may be reported as significant if the subject is dehydrated, and in contrast, proteinuria may be missed if the subject is overhydrated. In addition, the test readout could be altered depending on the test features (limit of detection and limit of quantification), alkalinity of the urine, and presence of infections. This is important, as proteinuria must be confirmed or excluded in subjects having kidney disease, e.g., subjects that have CKD or subjects that have received an allograft.

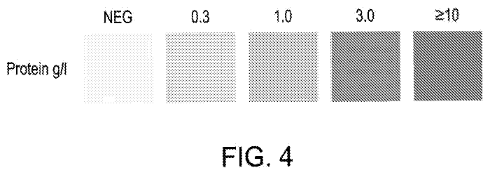

The present disclosure provides lateral flow device(s) for detecting the presence or absence of biomarkers, including one or more protein(s) in a biological sample. In some configurations the lateral flow device comprises a dye attached thereto which specifically binds to one or more protein(s) and/or a detectable moiety which specifically reacts with one or more protein(s), (e.g., albumin antibody). The threshold of detection of the one or more protein(s) can be adjusted depending on the application. In some instances a threshold of detection for forming a detectable one or more protein(s) complex or for dye detection of one or more proteins is at least 5 ng/ml, at least 10 ng/mL, at least 15 ng/mL, at least 20 ng/mL, at least 25 ng/ml, at least 30 ng/ml, at least 35 ng/mL, at least 40 ng/mL, at least 45 ng/mL, at least 50 ng/ml, at least 55 ng/ml, at least 60 ng/ml, at least 65 ng/ml, at least 70 ng/mL, at least 75 ng/ml, at least 80 ng/ml, at least 85 ng/mL, at least 90 ng/ml, at least 95 ng/ml, at least 100 ng/ml, at least 110 ng/ml, at least 120 ng/mL, at least 130 ng/mL, at least 140 ng/mL, at least 150 ng/mL, at least 160 ng/ml, at least 170 ng/mL, at least 180 ng/ml, at least 190 ng/ml, at least 200 ng/ml, at least 210 ng/ml, at least 220 ng/ml, at least 230 ng/mL, at least 240 ng/ml, or at least 250 ng/mL. In some instances, the detectable moiety is a color changing dye. The color changing dye may, in some instances, provide estimates for the amount of protein present in a sample as shown in FIG. 4. In some instances, the detectable moiety is albumin or another protein and an antibody is used for specifically forming a complex with albumin on a capture area.

The threshold of detection for the first detectable moiety can be adjusted depending on the application. In some instances, the first detectable moiety can become saturated. In some cases, a threshold of detection for forming a detectable one or more protein(s) complex is no more than 5 μg/mL, no more than 10 μg/mL, no more than 15 μg/mL, no more than μg/mL, no more than 25 μg/mL, no more than 30 μg/mL, no more than 35 μg/mL, no more than 40 μg/mL, no more than 45 μg/mL, no more than 50 μg/mL, no more than 55 μg/mL, no more than 60 μg/mL, no more than 65 μg/mL, no more than 70 μg/mL, no more than 75 μg/mL, no more than 80 g/mL, no more than 85 μg/mL, no more than 90 μg/mL, no more than 95 μg/mL, no more than 100 μg/mL, no more than 110 μg/mL, no more than 120 μg/mL, no more than 130 μg/mL, no more than 140 μg/mL, no more than 150 μg/mL, no more than 160 μg/mL, no more than 170 μg/mL, no more than 180 μg/mL, no more than 190 μg/mL, no more than 200 μg/mL, no more than 210 μg/mL, no more than 220 μg/mL, no more than 230 μg/mL, no more than 240 μg/mL, or no more than 250 μg/mL.

Detection of m-cIDNA

Small fragments of DNA circulate freely in the peripheral blood of healthy and diseased subjects. These cell-free DNA (cIDNA) molecules are thought to originate from dying cells and thus reflect ongoing cell death taking place in the body. In particular, cIDNA increases in kidney diseases during hemodialysis can indicate a proportion of kidney injury, e.g., molecular injury. Some results from cIDNA suggest that it is comparable to those from biopsies, the gold standard for measuring rejection for most transplanted organs. cIDNA on its own, however, remains a marker with insufficient sensitivity and specificity to identify kidney injury. Further, successful strategies for detection of cIDNA on lateral flow devices remain challenging.

Methylation patterns of circulating cell-free DNA (cIDNA) contain rich information about recent cell death events in the body. Aberrant DNA methylation has been described in chronic kidney disease (CKD). It has been reported that epigenetic mechanisms, one of which is DNA methylation, play a crucial role in the multiple biological events involved in post-transplant complications, such as alloimmune response, ischemia reperfusion injury (IRI), and kidney transplant graft fibrosis. In addition, studies have demonstrated a genome-wide DNA methylation pattern in inflamed renal tissue. Methylation occurs throughout the human genome and 5-methylcytosine is found in approximately 1.5% of human genomic DNA and is used by the present disclosure for detection of m-cIDNA.

An anti-m-cIDNA antibody can be added to a capture area on a lateral flow device on the disclosure and used for forming complexes with 5-methylcytosine in urine samples. 5-Methylcytosine is a methylated form of the DNA base cytosine (C) that regulates gene transcription and takes several other biological roles. The threshold of detection for them-cIDNA detectable moiety can be adjusted depending on the application. In some instances a threshold of detection for forming a detectable m-cIDNA complex is at least 5 ng/mL, at least 10 ng/ml, at least 15 ng/ml, at least 20 ng/ml, at least 25 ng/ml, at least 30 ng/mL, at least 35 ng/ml, at least 40 ng/ml, at least 45 ng/mL, at least 50 ng/mL, at least 55 ng/mL, at least 60 ng/ml, at least 65 ng/ml, at least 70 ng/mL, at least 75 ng/mL, at least 80 ng/ml, at least 85 ng/mL, at least 90 ng/ml, at least 95 ng/mL, at least 100 ng/mL, at least 110 ng/ml, at least 120 ng/mL, at least 130 ng/ml, at least 140 ng/ml, at least 150 ng/mL, at least 160 ng/ml, at least 170 ng/ml, at least 180 ng/ml, at least 190 ng/mL, at least 200 ng/mL, at least 210 ng/ml, at least 220 ng/mL, at least 230 ng/ml, at least 240 ng/ml, or at least 250 ng/ml.

The threshold of detection for the first detectable moiety can be adjusted depending on the application. In some instances, the first detectable moiety can become saturated. In some cases, a threshold of detection for forming a detectable m-cIDNA complex is no more than 5 μg/mL, no more than 10 μg/mL, no more than 15 μg/mL, no more than μg/mL, no more than 25 μg/mL, no more than 30 μg/mL, no more than 35 μg/mL, no more than 40 μg/mL, no more than 45 μg/mL, no more than 50 μg/mL, no more than 55 μg/mL, no more than 60 μg/mL, no more than 65 μg/mL, no more than 70 μg/mL, no more than 75 μg/mL, no more than 80 μg/mL, no more than 85 μg/mL, no more than 90 μg/mL, no more than 95 μg/mL, no more than 100 μg/mL, no more than 110 μg/mL, no more than 120 μg/mL, no more than 130 μg/mL, no more than 140 μg/mL, no more than 150 μg/mL, no more than 160 μg/mL, no more than 170 μg/mL, no more than 180 μg/mL, no more than 190 μg/mL, no more than 200 μg/mL, no more than 210 μg/mL, no more than 220 μg/mL, no more than 230 μg/mL, no more than 240 μg/mL, or no more than 250 μg/mL.

### Lateral Flow Device

A lateral flow device of the disclosure may be configured to identify a kidney injury, such as CKD or an allograft injury. In some configurations, the lateral flow device is configured to identifies an allograft rejection with at least 70%, at least 75%, at least 80%, at least 85%, or at least 90% sensitivity by providing a combined threshold of detection for forming a detectable m-cIDNA complex of at least 25 ng/mL and for forming a detectable m-cIDNA complex of at least 25 ng/ml. A lateral flow device of the disclosure may be configured to identify a kidney injury, such as CKD or an allograft injury. In some configurations, the lateral flow device is configured to identifies an allograft rejection with at least 70%, at least 75%, at least 80%, at least 85%, or at least 90% specificity by providing a combined threshold of detection for forming a detectable m-cIDNA complex of at least 25 ng/ml and for forming a detectable m-cIDNA complex of at least 25 ng/ml.

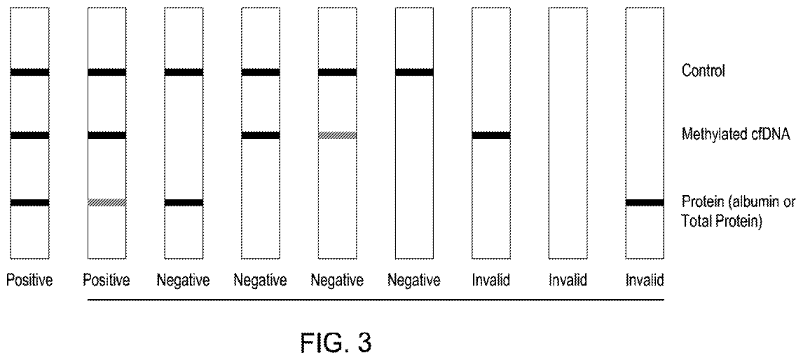

FIG. 3 depict an exemplary positive, negative, and invalid read-out of lateral flow devices for the detection of Kidney Transplant Rejection with the thresholds for detection set at 25 ng/ml for both m-cIDNA and one or more protein(s).

In some configurations, a lateral flow device of the disclosure comprises additional capture zones for capturing additional biomarkers. In some of these instances the capture area comprises a control detectable moiety, such as a species specific IgG (e.g., human or dog).

In some configurations, a first capture zone, a second capture zone, a third capture zones, and any number of suitable capture zones may extend the length of the test strip.

### Kits

Urinalysis home testing kits may comprise one or more lateral flow devices of the disclosure, containers configured to contain urine collection containers, including dipstick test reagent pads for measuring differing urinary properties, blot pads for removing excess urine from dipstick. Kits may comprise instructions for use of each component.

### Definitions

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

As used herein, “sensitivity” refers to a lateral flow device test ability to designate a subject with disease as positive. A highly sensitive test means that there are few false negative results, and thus fewer cases of disease are missed. The calculation for sensitivity is TP/(TP+FN), where TP is the total of true positives and FN is the total of false negatives; this fraction was expressed as a percentage.

As used herein, “specificity” refers to a lateral flow device test ability to designate a subject who does not have a disease as negative. The calculation for specificity was TN/(TN+FP), where TN is the total of true negatives and FP is the total of false positives; this fraction is also expressed as a percentage.

Note that as used herein and in the appended claims, the singular forms “a,” “an,” and “the” include plural referents unless the context clearly dictates otherwise. Thus, for example, reference to “a capture zone” refers to one or more capture zones, and reference to “sample receiving zone” includes reference to equivalent sample receiving zones, inlets, and the like known to those skilled in the art, and so forth. Additionally, it is to be understood that terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation. Furthermore, terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a lateral flow device involving detection of biomarker a, b, and c” means that the lateral flow device includes at least detection of biomarker a, b, and c.

Any numerical range recited herein includes all values and ranges from the lower value to the upper value. For example, if a concentration range is stated as 1% to 50%, it is intended that values such as 2% to 40%, 10% to 30%, 1% to 3%, or 2%, 25%, 39% and the like, are expressly enumerated in this specification. These are only examples of what is specifically intended, and all possible combinations of numerical values and ranges between and including the lowest value and the highest value enumerated are to be considered to be expressly stated in this application.

Numbers modified by the term “about” are intended to include +/−10% of the number modified.

As used herein, the terms “protein” and “polypeptide” are used interchangeably. Proteins may or may not be made up entirely of amino acids

### Example

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

### Identification of Biomarkers and Cut-Off Values for Development of a Rapid Test for Allograft Reiection

QSant™ utilizes a composite score of various biomarkers of distinct biochemical characteristics, i.e., proteins, metabolites, and nucleic acids. (See Yang, Sarwal, et al., A urine score for noninvasive accurate diagnosis and prediction of kidney transplant rejection. Science Translational Medicine, 18 Mar. 2020, Vol. 12, Issue 535). Yang et al. demonstrated that a urinary composite score of six biomarkers—an inflammation biomarker (e.g., CXCL-10, also known as IP-10); an apoptosis biomarker (e.g., clusterin); a cIDNA biomarkers; a DNA methylation biomarker; a creatinine biomarker; and total protein-enables diagnosis of Acute Rejection (AR), with a receiver-operator characteristic curve area under the curve of 0.99 and an accuracy of 96%. Notably, QSant™ (formerly known as QiSant™) predicts acute rejection before a rise in a stand-alone serum creatinine test, enabling earlier detection of rejection than currently possible by current standard of care tests.

The QSant™ results are returned in the form of a Q-Score, a Q-Score algorithm consisting of a trained machine learning linear model called a Random Forest (RF). A RF model is an aggregated predictive model and does not make any assumptions about distributions.

Therefore, it can accommodate different and variable degrees of skewness and tailedness in the individual biomarkers. A RF model utilizes these variations to associate non-fixed weights with different biomarkers. No single biomarker is responsible for determining an outcome for acute rejection (AR) or stable (NR); the Q-Score is calculated in aggregate. Analyte ranges are often overlapping and there is no universal range for NR vs AR. The QSant™ biomarkers have differential distributions in the NR and AR cohorts. Using a Kruskal Wallis test across balanced samples (N=38 per cohort), statistically significant differences are observed for some biomarkers but not all. The distributions are often skewed with a high degree of kurtosis, specifically heavy right tails.

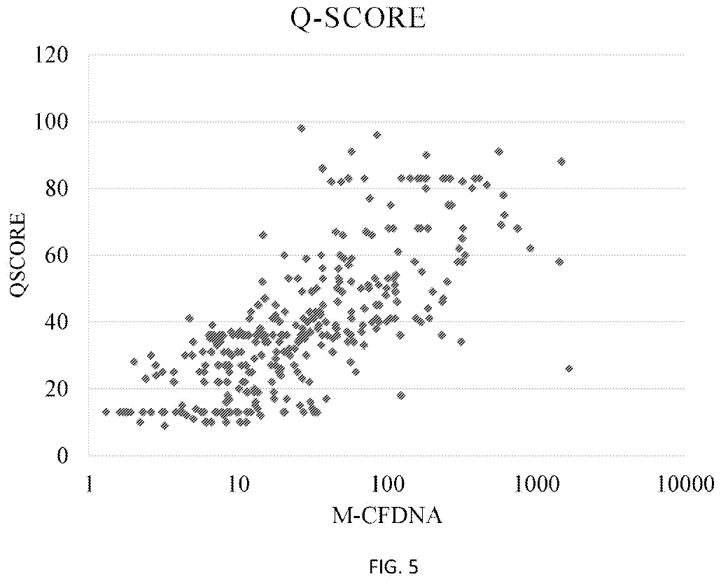

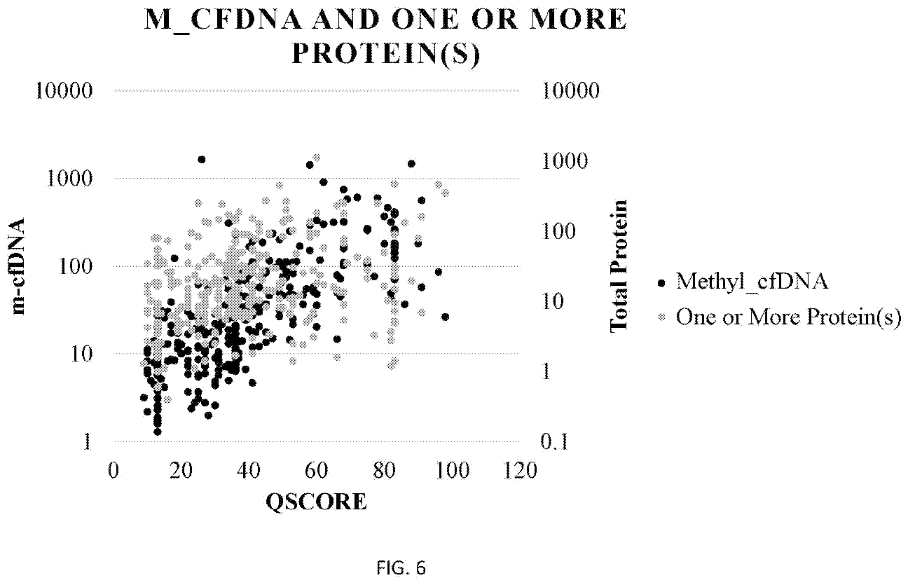

The QSant™ performance was tested on 228 individual that participated on an early access study, the AQUA registry. The AQUA registry is a multi-center observational study to assess the clinical management of kidney transplant recipients (KTRs) with use of the QSant™ test. The total levels of analyte detected are described on TABLE 1. FIG. 5 is a plot of them-cIDNA values vs. the Q-Score for each sample. FIG. 6 is a log-linear plot of m-cIDNA values (blue) and total protein values (orange) vs. the Q-Score for each sample.

TABLE 1

| | Days | | | | | | |
|---|---|---|---|---|---|---|---|
| ID | P̅T | CIDNA | m cIDNA | CXCL10 | Clusterin | Creatinine | Total Protein |
| P0002N-03 | 6355 | 722.5 | 9 | 6.4 | 113.8 | 63.3 | 10.6 |
| P00071-0l | 321 | 6508 | 82.6 | 72.4 | 271.5 | 103.8 | 9.1 |
| P0004W-0l | 1377 | 19.3 | 1.6 | 0.3 | 26.5 | 22.1 | 3.5 |
| P0003W-02 | 153 | 1094.4 | 13.5 | 24.8 | 157.4 | 26.4 | 38.8 |
| P0005R-0l | 5401 | 4465.6 | 47.7 | 11.9 | 456.2 | 68.9 | 151.7 |
| P0004O-02 | 126 | 353.6 | 3.2 | 2.3 | 31 | 21.5 | 1.3 |
| P00046-02 | 545 | 340.3 | 2.8 | 25.5 | 227.1 | 46.3 | 12.7 |
| P0005M-0l | 81 | 30911.2 | 317.6 | 38.1 | 425.3 | 100.2 | 15.8 |
| P00058-02 | 273 | 3130.4 | 152.4 | 22.9 | 84.6 | 47.3 | 5.8 |
| P00061-0l | 389 | 358.3 | 7.3 | 3.1 | 113.6 | 60.9 | 37.7 |
| P00060-0l | 539 | 763.7 | 17.4 | 0.1 | 29.5 | 27.7 | 6.3 |
| P0000U-02 | 1721 | 6118 | 49.1 | 0.8 | 47.6 | 29.3 | 3.7 |
| P000lU-05 | 6459 | 1286.1 | 11.9 | 17.3 | 100.7 | 46.9 | 29.7 |
| P00036-02 | 198 | 341.4 | 11.4 | 7.8 | 179.9 | 87 | 18.7 |
| P0003X-02 | 351 | 88.3 | 1.7 | 5.9 | 54.1 | 28.7 | 3.5 |
| P00066-0l | 3936 | 2007.8 | 26 | 24.8 | 211.9 | 117 | 55.7 |
| P00065-0l | 58 | 460.1 | 8.7 | 13.6 | 157.3 | 59.9 | 20.2 |
| P00067-0l | 80 | 1343.3 | 39 | 111.5 | 286.3 | 99.6 | 26.3 |
| P00069-0l | 32 | 5926.6 | 97.8 | 14.8 | 502.6 | 77.6 | 29.7 |
| P0006A-0l | 215 | 17632 | 180 | 606.8 | 1684.6 | 90.6 | 469.2 |
| P00027-03 | 2499 | 18787.3 | 182.3 | 595.2 | 1268.4 | 33.6 | 79.1 |
| P0006B-0l | 1748 | 2851.6 | 27.9 | 19.1 | 170.2 | 107.2 | 13.1 |
| P0006C-0l | 1628 | 6039.9 | 36.6 | 28.9 | 1136.4 | 190.6 | 98.4 |
| P0002V-02 | 6317 | 6032.6 | 910.6 | 20.3 | 97.3 | 73.5 | 17.2 |
| P0003S-02 | 962 | 30.2 | 9.7 | 27.3 | 99.1 | 28.8 | 7.1 |
| P00062-0l | 4927 | 17164.3 | 562.3 | 575.2 | 547 | 80.3 | 161.7 |
| P0004O-03 | 154 | 277.3 | 5 | 9.5 | 131.3 | 32.7 | 10.2 |
| P0005H-02 | 343 | 2858.3 | 46.2 | 48.3 | 238.1 | 59.2 | 8 |
| P0003 l-02 | 1917 | 209.8 | 5.7 | 1.7 | 26 | 28.4 | 2.5 |
| P0005P-0l | 75 | 4478.3 | 14.7 | 45.7 | 38.9 | 52.6 | 232 |
| P0005N-0l | 75 | 633.4 | 2 | 11.7 | 24.9 | 70.1 | 131.8 |
| P0005O-0l | 75 | 7833.7 | 26.5 | 81.2 | 54.7 | 35.5 | 349.8 |
| P0005T-0l | 1461 | 6572.1 | 85.4 | 10.8 | 8085.3 | 59.1 | 462.8 |
| P0005V-0l | 174 | 5157.7 | 51.2 | 14.8 | 96.9 | 39.5 | 27.5 |
| P0004J-02 | 849 | 3698.3 | 21.9 | 34.8 | 837.4 | 132.9 | 242.8 |
| P0005U-0l | 475 | 2241.4 | 26.8 | 10.1 | 104 | 49.6 | 15.8 |
| P000IQ-02 | 5310 | 968.7 | 10 | 20 | 65.1 | 96.1 | 22.1 |
| P000IG-02 | 3668 | 27337.6 | 263 | 8.5 | 58.3 | 44.2 | 32 |
| P0005Y-0l | 128 | 13117.7 | 232.4 | 236 | 472.6 | 143.1 | 27.8 |
| P0005W-0l | 57 | 9978.7 | 118.2 | 100.5 | 713.9 | 92.6 | 23.4 |
| P0005X-0l | 440 | 21180 | 182.8 | 35.2 | 355 | 77.5 | 32.3 |
| P0002N-02 | 6286 | 79 | 17.1 | 20.8 | 334.8 | 88.1 | 39.6 |
| P0005Z-0l | 15 | 11260.6 | 180.9 | 22 | 381.3 | 80 | 49.8 |
| P0005H-0l | 309 | 23.3 | 13.1 | 40.3 | 263 | 79.9 | 6.7 |
| P00008-03 | 1822 | 6812.4 | 170.2 | 32.6 | 167 | 130.7 | 12 |
| P0003Q-05 | 100 | 2227.2 | 56.8 | 36.8 | 218.7 | 30.8 | 13.7 |
| P0002C-02 | 4304 | 2.7 | 7.3 | 5.2 | 82 | 43.9 | 8.2 |
| P00026-02 | 6765 | 130 | 3.1 | 2.5 | 23.9 | 29.9 | 2.6 |
| P0003Q-03 | 76 | 14600.6 | 1473.8 | 438 | 454.1 | 48.9 | 19.5 |
| P0005J-0l | 473 | 10680.3 | 370.8 | 13.2 | 163.3 | 67 | 16.5 |
| P0002B-02 | 3211 | 18657.9 | 1431.3 | 243 | 378.3 | 131.2 | 96.7 |
| P0005I-0l | 5214 | 4663.9 | 43.4 | 26 | 172 | 115.1 | 10.6 |
| P0002J-03 | 216 | 724.4 | 12.3 | 4.1 | 161.5 | 102.5 | 31 |
| P0005K-0l | 6314 | 270.6 | 6.6 | 12.2 | 100.2 | 67.1 | 7.9 |
| P000IS-02 | 6271 | 1725 | 9.9 | 12.4 | 94.1 | 123.8 | 36.2 |
| P0004F-02 | 473 | 2626.9 | 45 | 66.2 | 499.4 | 69.2 | 254.8 |
| P00063-0l | 303 | 755.3 | 8.3 | 5.4 | 215.9 | 30.6 | 73.2 |
| P00052-02 | 848 | 1146.1 | 9 | I.I | 39.8 | 17.3 | 3.6 |
| P00064-0l | 1611 | 460.6 | 26.3 | 29.2 | 211 | 89.6 | 9.3 |
| P0007J-0l | 2295 | 528.8 | 8.5 | 21.7 | 236.3 | 40.1 | 17.8 |
| P0005T-02 | 1538 | 3046.9 | 36.8 | 3.8 | 531.1 | 24.1 | 133.1 |
| P0007K-0l | 2807 | 33.7 | 2.6 | 8.5 | 172.1 | 79.6 | 7.1 |
| P0007L-0l | 373 | 47.5 | 6.4 | 15.4 | 288.2 | 94.8 | 15.9 |
| P0007M-0l | 508 | 445.5 | 49.7 | 11.5 | 142.5 | 127.5 | 8.2 |
| P0006E-02 | 99 | 1032.8 | 11.3 | 24.9 | 224.6 | 86 | 34.5 |
| P00021-0l | 7694 | 2295 | 57.4 | 22 | 354.8 | 2.2 | 6.9 |
| P00059-02 | 169 | 180.8 | 2.2 | 3.1 | 63.9 | 41.5 | 2.7 |
| P0007O-0l | 2721 | 1005.1 | 8.6 | 62.9 | 214.3 | 149.8 | 12 |
| P0002V-04 | 2817 | 438.8 | 156.7 | 18.7 | 112.9 | 70.4 | 17.3 |
| P0007P-0l | 1126 | 152.2 | 6.7 | 30.3 | 252.7 | 104 | 26 |
| P0007Q-0l | 600 | 7853.7 | 112.6 | 29.2 | 415.9 | 191.2 | 26.1 |
| P0007R-0l | 7021 | 2303.3 | 20.3 | 18.5 | 2628.2 | 56.6 | 183 |
| P0007T-0l | 1251 | 1024.8 | 10.9 | 9.3 | 109.3 | 81.8 | 24.9 |
| P0007S-0l | 40 | 1493.2 | 14.3 | 17.4 | 226 | 79 | 19 |
| P00004-0l | 21 | 2532.9 | 36.9 | 69.6 | 511.1 | 49.3 | 30.6 |
| P00005-0l | 1446 | 275.8 | 8.1 | 8.4 | 150.1 | 59 | 6.7 |
| P00007-0l | 1045 | 2779.5 | 112.4 | 110.8 | 1503.1 | 91.8 | 147.2 |
| P00006-0l | 303 | 663.2 | 17.7 | 24.4 | 654.1 | 113.2 | 78.6 |
| P00008-0l | 1601 | 694.7 | 13.6 | 4.2 | 28.5 | 39.8 | 3.7 |
| P0000D-01 | 3904 | 1747.2 | 45 | 34.7 | 561 | 96 | 91.1 |
| P0000B-01 | 320 | 6948.7 | 78.7 | 40.9 | 178.8 | 74.1 | 14.5 |
| P0000C-01 | 2912 | 1489.1 | 37 | 32.4 | 1906.5 | 115 | 272.6 |
| P0000A-01 | 637 | 182.2 | 3.1 | 26 | 60.5 | 60.4 | 8.4 |
| P0000F-01 | 870 | 543.9 | 11.5 | 8.2 | 75.1 | 25.4 | 6.7 |
| P0000E-01 | 334 | 450.2 | 19.9 | 77.7 | 366.2 | 63.5 | 29.1 |
| P00002-0l | 2192 | 688.2 | 34.6 | 35.1 | 397 | 128.2 | 30.7 |
| P00003-0l | 37 | 675.3 | 32.3 | 36 | 629 | 114.8 | 80.3 |
| P0000P-01 | 5027 | 4007.7 | 111.1 | 11.2 | 505.2 | 103.7 | 103.5 |
| P0000O-01 | 3333 | 171.2 | 4.9 | 41.8 | 80.4 | 75.2 | 21.2 |
| P0000Q-01 | 153 | 1123.8 | 25.9 | 13.1 | 272.2 | 196.1 | 34.6 |
| P0000R-01 | 1067 | 354.6 | 11.7 | 32.6 | 73 | 72.6 | 9.9 |
| P0000S-01 | 922 | 2724.8 | 39.7 | 24.8 | 197.1 | 175.3 | 6 |
| P0000U-01 | 1514 | 6059.2 | 160.2 | 8.3 | 30.4 | 20.2 | 1.4 |
| P00004-02 | 56 | 2714.2 | 36 | 24.6 | 260.5 | 73.9 | 26.4 |
| P0000W-01 | 1980 | 211.4 | 6.9 | 8.8 | 122 | 88.6 | 14.5 |
| P0000X-01 | 1628 | 446.3 | 18 | 57.1 | 510.9 | 198.8 | 152 |
| P0000V-01 | 540 | 321.5 | 14.4 | 21.2 | 3238.4 | 71.7 | 142.1 |
| P0000Y-01 | 2158 | 3081.1 | 257.6 | 10.2 | 101.5 | 19.5 | 21 |
| P0000Z-01 | 6 | 2150.8 | 33.5 | 8.9 | 108.9 | 67.2 | 19.6 |
| P0000B-02 | 344 | 1200.1 | 31.5 | 16.9 | 178.8 | 65.3 | 30.6 |
| P0000C-02 | 2940 | 594.1 | 20.6 | 5.6 | 136.1 | 52 | 45.9 |
| P0000M-02 | 202 | 2047.1 | 28.6 | 1.5 | 20.4 | 31.8 | 2.5 |
| P00012-0l | 740 | 1268.8 | 17 | 17.3 | 260.5 | 60.4 | 10.2 |
| P000ll-01 | 648 | 221.6 | 6.6 | 45.9 | 140.5 | 80.1 | 10.6 |
| P000I0-01 | 3446 | 505.4 | 10.3 | 40.6 | 94.5 | 92.8 | 15.1 |
| P000IV-02 | I | 2295 | 57.4 | 22 | 354.8 | 42.2 | 6.9 |
| P00013-0l | 1727 | 705.8 | 13.6 | 10.5 | 382.6 | 147.7 | 8.3 |
| P000IV-03 | 2650 | 2295 | 57.4 | 22 | 354.8 | 99.2 | 6.9 |
| P00016-02 | 90 | 355.4 | 12.1 | 3 | 90.4 | 41.5 | 16.4 |
| P000IA-01 | 16 | 8182 | 142.3 | 4.1 | 127.7 | 6 | 15.1 |
| P000I7-01 | 31 | 1411.7 | 27.7 | 10.6 | 150.5 | 76.1 | 12 |
| P00019-0l | 54 | 830.4 | 12.8 | 8.5 | 136.6 | 52.6 | 8.3 |
| P00015-0l | 75 | 146.1 | 6.6 | 0.1 | 43.9 | 35.5 | 7.4 |
| P00018-0l | 65 | 4881.2 | 76.2 | 0.1 | 46.2 | 22.4 | 3.3 |
| P00014-0l | 21 | 1265.8 | 24.3 | 16.5 | 78 | 47.2 | 6.9 |
| P000IC-01 | 45 | 688.5 | 10.1 | 0.1 | 47.5 | 37.8 | 4.8 |
| P000IB-01 | 27 | 4482.9 | 54.8 | 0.2 | 50.9 | 17.8 | 7.2 |
| P000ID-01 | 63 | 2079.8 | 22.2 | 0.3 | 32.8 | 32.4 | 3.5 |
| P000I V-01 | I | 2295 | 57.4 | 22 | 354.8 | 42.2 | 6.9 |
| P0000H-01 | 559 | 291.6 | 8.7 | 4.6 | 78.6 | 59.2 | 2.5 |
| P00009-02 | 3480 | 3096.7 | 75.2 | 46.3 | 130.2 | 40 | 19.1 |
| P0000G-01 | 577 | 4152 | 84.2 | 59.8 | 522.8 | 137.9 | 50.1 |
| P000IV-06 | 2652 | 2295 | 57.4 | 22 | 354.8 | 2.2 | 6.9 |
| P0000K-01 | 2867 | 189.5 | 23.7 | 110.9 | 718.3 | 48.3 | 137.8 |
| P000OI-01 | 1001 | 2170.9 | 85 | 32.2 | 492.2 | 161.9 | 52.6 |
| P0000J-01 | 204 | 149.8 | 7.2 | 25.7 | 187.9 | 39.9 | 2.6 |
| P0000L-01 | 1930 | 1929.2 | 27.4 | 27.1 | 93.1 | 77.6 | 3 |
| P0000M-01 | 184 | 2349.6 | 42 | 0.1 | 7 | 12.4 | 1.2 |
| P0000N-01 | 1242 | 1688.8 | 35.4 | 46.2 | 443.8 | 170.5 | 41.5 |
| P000IJ-01 | 5131 | 3331.3 | 186.8 | 28.8 | 589.6 | 35.9 | 178.8 |
| P00005-02 | 1488 | 835 | 17.3 | 1.6 | 62.7 | 35.2 | 5.4 |
| P0000O-02 | 3354 | 108.9 | 4.1 | 6.4 | 105.2 | 34.1 | 43.5 |
| P000IG-01 | 3497 | 12211.7 | 244.6 | 9.7 | 103.5 | 41 | 27.9 |
| P000IH-01 | 3950 | 5768.6 | 322.4 | 30 | 224.1 | 50.7 | 68.5 |
| P000II-01 | 1208 | 12150.5 | 269.2 | 18.8 | 177.3 | 79.9 | 27.8 |
| P000IV-13 | 2663 | 2295 | 57.4 | 17.7 | 22.3 | 196.1 | 99.8 |
| P000IM-02 | 3817 | 235.3 | 5.9 | 17.5 | 42 | 62 | 6.4 |
| P000IP-01 | 54 | 246.6 | 6.1 | 4.7 | 32.9 | 21.6 | 4.2 |
| P000IQ-01 | 5153 | 5121.5 | 59.5 | 47.2 | 97 | 144.1 | 36.7 |
| P000IV-08 | 2655 | 2295 | 57.4 | 22 | 15.1 | 20.1 | 6.9 |
| P000IS-01 | 6130 | 3716.4 | 47.3 | 3.2 | 20.1 | 37.2 | 8.9 |
| P000IL-03 | 918 | 261.6 | 7 | 3 | 74.7 | 22.5 | 17.1 |
| P000IR-01 | 1261 | 3457 | 113.3 | 1.5 | 68.7 | 62.8 | 18.1 |
| P000IO-02 | 706 | 6254.5 | 105 | 6 | 323.1 | 31.9 | 38.2 |
| P000IU-01 | 6289 | 1524.6 | 17.6 | 8.1 | 81.1 | 49.9 | 28.7 |
| P000IZ-01 | 878 | 20497.7 | 415 | 29.2 | 27.2 | 79.7 | 29.7 |
| P0000T-02 | 3330 | 2853.7 | 55 | 29.9 | 44.1 | 49 | 5.1 |
| P000I T-01 | 346 | 354.9 | 8.5 | 11.9 | 1538.2 | 24.2 | 55.2 |
| P000IX-01 | 1434 | 4240.3 | 43 | 32.5 | 238.6 | 131.1 | 13.4 |
| P000I Y-01 | 2565 | 5332.9 | 113.9 | 31.1 | 726.7 | 66.6 | 47.6 |
| P0000Z-02 | 46 | 1173 | 11.3 | 3.4 | 82.3 | 89.3 | 10.5 |
| P000IW-01 | 7682 | 1588.2 | 16.6 | 11.8 | 12.7 | 33 | 5.3 |
| P000IV-09 | 2656 | 2295 | 57.4 | 22 | 15.1 | 20.9 | 6.9 |
| P00020-0l | 1372 | 37.4 | 1.3 | 0.1 | 18.4 | 25.1 | 1.6 |
| P000IN-01 | 2159 | 3794.5 | 167.6 | 208.2 | 545.8 | 152.3 | 87.9 |
| P000IO-01 | 690 | 554.4 | 21.7 | 0.1 | 4.9 | 5.9 | 1.4 |
| P000I V-12 | 2661 | 1295 | 57.4 | 20 | 354.8 | 44.1 | 1.8 |
| P00022-0l | 5878 | 2922.3 | 66.7 | 3.7 | 365.3 | 39 | 83.8 |
| P00024-0l | 3996 | 0.5 | 5.8 | 7.2 | 127.2 | 69.3 | 7.9 |
| P00025-0l | 2042 | 875.7 | 46.1 | 39.1 | 228.8 | 134.5 | 155.7 |
| P00028-0l | 3032 | 171.5 | 7.6 | 6.3 | 177.7 | 81.6 | 31.9 |
| P00026-0l | 6650 | 51.8 | 2.4 | 10.2 | 78.8 | 52 | 3.9 |
| P00027-0l | 2331 | 0.2 | 18.1 | 6.9 | 204.5 | 75.6 | 6.3 |
| P0002D-0l | 4074 | 9726.6 | 319.7 | 0.5 | 31 | 27.7 | 2.8 |
| P0002G-0l | 362 | 92.1 | 2.8 | 119.8 | 15 | 14.1 | I.I |
| P0000Z-03 | 60 | 6028.6 | 68.3 | 44.5 | 146.6 | 73.9 | 26.5 |
| P0002B-0l | 3094 | 500.3 | 19 | 69.9 | 89.2 | 48.4 | 10.6 |
| P0002A-0l | 5018 | 150.6 | 18.8 | 131.6 | 132 | 80 | 35.9 |
| P0002E-0l | 2437 | 677.2 | 11.1 | 4.7 | 80.8 | 42.5 | 15.2 |
| P0002F-0l | 7467 | 617.9 | 13 | 26 | 19.6 | 35.3 | 0.4 |
| P0002C-0l | 4191 | 9.5 | 6.5 | 20.1 | 89.9 | 71.5 | 8.4 |
| P0000T-03 | 3348 | 1454.1 | 38.7 | 17.3 | 28.1 | 41.5 | 5.9 |
| P0002O-0l | 985 | 1571.3 | 32.1 | 32.7 | 196.9 | 63.7 | 55.8 |
| P0000G-02 | 651 | 1780 | 28.6 | 5.4 | 87.4 | 15.6 | 3.1 |
| P0002I-0l | 10117 | 746.4 | 87.5 | 117.2 | 462.7 | 87.3 | 269 |
| P0002K-0l | 691 | 814.7 | 12.2 | 62.2 | 137.4 | 109.5 | 22.1 |
| P0002H-0l | 4149 | 123.8 | 17.8 | 7.6 | 99.5 | 62 | 8.5 |
| P0002J-0l | 104 | 291.9 | 8 | 4.2 | 80 | 68.6 | 18.5 |
| P0002L-0l | 2602 | 72.6 | 3.8 | 2.4 | 26.6 | 30.4 | 4.9 |
| P0002M-0l | 7011 | 1589.3 | 28.6 | 16.4 | 174.1 | 85.4 | 8.8 |
| P0000Z-04 | 67 | 1073.3 | 7.4 | 2.5 | 49.9 | 43.9 | 10.6 |
| P0002N-0l | 6182 | 98.5 | 3.2 | 2.1 | 10.9 | 22.1 | 1.9 |
| P0002R-0l | 160 | 8232.7 | 332.7 | 22.7 | 133.6 | 74.7 | 18 |
| P0002P-0l | 598 | 896.6 | 116.1 | 1.3 | 18.9 | 13.1 | 3.4 |
| P0002G-02 | 375 | 68.3 | 7.4 | 8.3 | 62.5 | 49.3 | 5 |
| P0002S-0l | 1710 | 11554.4 | 600.7 | 48.5 | 916.4 | 82.2 | 250.6 |
| P0002T-0l | 1934 | 1233 | 19.5 | 8.9 | 151.2 | 85.1 | 11.2 |
| P0002U-0l | 155 | 834 | 20.2 | 4.8 | 43.1 | 27.7 | 6.8 |
| P0002V-0l | 2647 | 13706 | 296.4 | 52.4 | 281.2 | 121.5 | 81.7 |
| P00023-0l | 297 | 624.2 | 7.1 | 77.4 | 136.3 | 297.5 | 46 |
| P000lU-02 | 6324 | 752.5 | 13 | 0.2 | 42.8 | 27.5 | 35.5 |
| P00033-0l | 355 | 620.3 | 13.8 | 20.4 | 155.1 | 56.7 | 49.1 |
| P00007-02 | 1155 | 843.6 | 17.8 | 53.2 | 228.1 | 170 | 46 |
| P00029-02 | 1477 | 142.8 | 8.4 | 64.2 | 474 | 196.1 | 60.2 |
| P00034-0l | 5860 | 386.9 | 30.1 | 18.3 | 453.2 | 41.4 | 127.6 |
| P00035-0l | 86 | 43.1 | 6 | 8.8 | 70.4 | 27.3 | 6.8 |
| P0000B-03 | 426 | 4931.3 | 45.4 | 54.1 | 491.5 | 108.6 | 22.2 |
| P00037-0l | 183 | 729.1 | 14.1 | 101.7 | 529.7 | 130.8 | 80.6 |
| P00036-0l | 73 | 0.7 | 8.3 | 10.5 | 291.4 | 85.2 | 16.7 |
| P00038-0l | 3096 | 133.2 | 5.9 | 62.1 | 334 | 31.2 | 32.8 |
| P0003B-0l | 1054 | 7299.2 | 236.1 | 6.2 | 36.1 | 40.8 | 9.5 |
| P0003A-0l | 150 | 457.9 | 33.8 | 26.8 | 164.6 | 112 | 18.6 |
| P0003D-0l | 376 | 5538.4 | 74.5 | 14.5 | 1498 | 100 | 202.8 |
| P0003C-0l | 241 | 450.9 | 6.7 | 52.9 | 197.8 | 71.1 | 31.2 |
| P0003E-0l | 514 | 2298.3 | 20.3 | 41.4 | 258.5 | 206.9 | 17.3 |
| P00039-02 | 1693 | 1802.5 | 27 | 137.7 | 333.3 | 208 | 32 |
| P0002X-02 | 1293 | 1974.3 | 67 | 5.4 | 79.7 | 25.9 | 7.2 |
| P0003F-0l | 915 | 5742.6 | 70.2 | 5.1 | 199.7 | 111.4 | 27.1 |
| P00009-03 | 3588 | 1378.8 | 17.8 | 99.8 | 275.6 | 35.1 | 44 |
| P0002Z-0l | 2804 | 898.7 | 8 | 19.8 | 915 | 152.5 | 146.2 |
| P0002W-0l | 143 | 999 | 20.5 | 19.1 | 189.7 | 104.4 | 32 |
| P0003I-0l | 243 | 388.3 | 7.2 | 6.3 | 69.5 | 54 | 5.5 |
| P0003H-0l | 100 | 3516.4 | 50.4 | 14 | 25.7 | 24.2 | 1.6 |
| P000lZ-02 | 914 | 9569 | 579.7 | 193.5 | 125.6 | 68.9 | 35 |
| P0002Y-0l | 70 | 11387.8 | 610 | 118.3 | 360.5 | 63.3 | 42.7 |
| P00030-0l | 4286 | 15.1 | 3.7 | 8 | 53.1 | 48.9 | 3.2 |
| P000lJ-02 | 5221 | 930.3 | 30.4 | 46.1 | 498.1 | 81.1 | 189.1 |
| P0003N-0l | 1651 | 4159.9 | 187.3 | 47.7 | 299.1 | 134.1 | 19.9 |
| P0003J-0l | 1408 | 38.4 | 8.7 | 4.3 | 91.2 | 43.8 | 5 |
| P0003G-02 | 1756 | 1871.1 | 234.3 | 6.6 | 115.3 | 64.8 | 14.3 |
| P0003K-0l | 1369 | 1172.2 | 23.8 | 5.7 | 118.2 | 39.8 | 19.4 |
| P0003M-0l | 2683 | 2643 | 79 | 19.7 | 311.8 | 121.8 | 16.6 |
| P0002Q-02 | 390 | 740.4 | 123.7 | 10.6 | 52.7 | 27.3 | 6.3 |
| P00032-0l | 81 | 362.3 | 10.3 | 14.6 | 234.8 | 85.5 | 13 |
| P0003 l-0l | 1819 | 0.5 | 3.7 | 5.5 | 68.8 | 55.3 | 4.9 |
| P0006H-0l | 1516 | 77 | 1.9 | 1.5 | 23.7 | 20.4 | 0.6 |
| P0006F-0l | 745 | 152.2 | 2.3 | 16.1 | 21.7 | 40.1 | 2.4 |
| P00057-02 | 1608 | 21833.8 | 388.6 | 7.4 | 130.2 | 36.9 | 4.9 |
| P0006I-0l | 1607 | 268.8 | 6 | 11.3 | 216.6 | 58.4 | 9.1 |
| P0006K-0l | 618 | 16512.1 | 318 | 310.5 | 3245.3 | 124.3 | 132.9 |
| P0006E-0l | 68 | 16532.6 | 312.3 | 235.1 | 506.6 | 211.6 | 170.5 |
| P0006G-0l | 3822 | 929.4 | 15.1 | 9.5 | 97.8 | 76.9 | 23.6 |
| P0006L-0l | 1192 | 645.6 | 14.1 | 22.4 | 105.3 | 52.4 | 5.9 |
| P0002V-03 | 2791 | 8743.2 | 124.1 | 14.2 | 91.2 | 53.1 | 23 |
| P0006M-0l | 735 | 5522.6 | 109.9 | 36.1 | 190.6 | 51.6 | 8.9 |
| P0006N-0l | 198 | 846.4 | 11.8 | 15.5 | 166 | 56.4 | 6.6 |
| P0006O-0l | 687 | 7016.4 | 88.3 | 61.4 | 297.8 | 119.8 | 45.4 |
| P0002B-03 | 3278 | 12621.3 | 167.9 | 160.1 | 431.9 | 74.2 | 90.9 |
| P0006R-0l | 1616 | 2435.1 | 36.8 | 3.4 | 42.2 | 43 | 2.4 |
| P0006X-0l | 2600 | 973.8 | 10.5 | 32.5 | 138.7 | 166.8 | 13.4 |
| P0006Q-0l | 9348 | 2399.8 | 31.2 | 19.2 | 57.9 | 52.3 | 10.9 |
| P0006S-0l | 194 | 994.6 | 18.8 | 8.8 | 537.4 | 98.3 | 253.6 |
| P0006W-0l | 226 | 221.9 | 4.2 | 3.6 | 27.2 | 27.7 | 5 |
| P0006Y-0l | 196 | 911.8 | 16.8 | 69.5 | 478.9 | 147.9 | 35.4 |
| P0006T-0l | 294 | 1684.7 | 15.7 | 0.3 | 34 | 42.9 | 7 |
| P0006V-0l | 751 | 84.7 | 10.7 | 6.3 | 105.4 | 43.2 | 12 |
| P0007B-0l | 741 | 4028.5 | 48.3 | 19.4 | 169.7 | 53.5 | 93.6 |
| P0007A-0l | 747 | 9976 | 201.2 | 154 | 1201 | 194.6 | 450 |
| P0006C-02 | 1647 | 1110.5 | 11.1 | 11.1 | 870.5 | 94.6 | 70.6 |
| P0006Z-0l | 307 | 6424.3 | 57 | 89.6 | 715.3 | 153.8 | 22.4 |
| P00072-0l | 238 | 2613.9 | 25 | 76.1 | 479.9 | 105.9 | 84.4 |
| P0006P-02 | 168 | 1049.4 | 11.5 | 4.8 | 131.5 | 53.1 | 7.2 |
| P0003P-0l | 3926 | 2001.8 | 29.1 | 10.6 | 82.8 | 91.8 | 10.4 |
| P00009-04 | 3613 | 906.5 | 30.5 | 24.5 | 108.8 | 23.9 | 25.7 |
| P0000M-03 | 313 | 3847.7 | 252.3 | 1.6 | 51.2 | 70.5 | 6.5 |
| P00012-02 | 851 | 21.8 | 30.1 | 12.2 | 227.8 | 52.5 | 9.1 |
| P0003R-0l | 117 | 91.8 | 8.8 | 0.1 | 57.4 | 9.5 | 0.6 |
| P0003X-0l | 258 | 135.6 | 14.5 | 59.6 | 269.1 | 103.1 | 10.9 |
| P0003S-0l | 881 | 58.2 | 4.2 | 0.1 | 24.6 | 10.5 | I.I |
| P0003T-0l | 860 | 63.9 | 10 | 20.6 | 65.2 | 32.5 | 9.4 |
| P0003W-0l | 111 | 863.9 | 27.4 | 30.3 | 84.6 | 22.5 | 41.8 |
| P0003U-0l | 4560 | 9055.2 | 748.1 | 21.3 | 77.5 | 75.1 | 13.6 |
| P0003Y-0l | 3007 | 115.9 | 15.1 | 13.9 | 98.7 | 108.6 | 29.9 |
| P0003V-0l | 4202 | 700.8 | 35.7 | 15.7 | 162.3 | 155.2 | 32.6 |
| P0000C-03 | 3053 | 258.3 | 99.1 | 25.6 | 375 | 149.6 | 135.7 |
| P0000H-02 | 694 | 1959.1 | 87.6 | 7.7 | 243.9 | 148.4 | 8.9 |
| P0000O-03 | 3460 | 79.2 | 5.9 | 3.8 | 55.5 | 45.6 | 20.6 |
| P00040-0l | 5446 | 120 | 13.3 | 18.6 | 291.8 | 176.5 | 44 |
| P0003Z-0l | 64 | 279.5 | 31.5 | 45 | 91.8 | 45.3 | 6.7 |
| P0003N-02 | 1667 | 842.6 | 53.8 | 8.8 | 46.6 | 58.4 | 6.3 |
| P00042-0l | 2514 | 1690.5 | 104.6 | 10.1 | 136.7 | 80 | 11.2 |
| P00041-0l | 276 | 1666.9 | 99.1 | 18.5 | 468.7 | 92 | 16.4 |
| P0000J-02 | 340 | 17.3 | 7.2 | 0.1 | 40.5 | 23.5 | 3.6 |
| P00043-0l | 1842 | 1781.9 | 33.1 | 19.4 | 802.2 | 182.8 | 10.4 |
| P00044-0l | 4057 | 1669.8 | 84.5 | 13.8 | 179.5 | 110.6 | 14.4 |
| P0000L-02 | 2067 | 18.8 | 5.9 | 0.1 | 14.9 | 26.9 | I |
| P000I 1-02 | 765 | 5.1 | 1.8 | 8 | 17.5 | 31.3 | 2.3 |
| P0003Q-02 | 28 | 606.7 | 69.7 | 68.3 | 433.9 | 16.2 | 33.4 |
| P0002M-02 | 7055 | 4159.9 | 187.3 | 47.7 | 299.1 | 134.1 | 19.9 |
| P00049-0l | 2079 | 225 | 54.3 | 16.5 | 74.1 | 76.3 | 11.2 |
| P00070-0l | 227 | 25658.3 | 465.3 | 8.5 | 67.4 | 23.3 | 10.3 |
| P0005J-02 | 546 | 706.7 | 7.5 | 21 | 534 | 116.4 | 42.2 |
| P0006J-02 | 4416 | 439 | 9.2 | 6 | 84.5 | 45.2 | 1.7 |
| P0006U-02 | 901 | 619.6 | 8.3 | 3.2 | 56.2 | 12.7 | 1.4 |
| P00073-0l | 231 | 4201.8 | 61.4 | 10.3 | 364.8 | 119.5 | 19.6 |
| P00075-0l | 71 | 1342.8 | 26.9 | 85 | 230.2 | 73.2 | 94.9 |
| P00078-0l | 367 | 915.4 | 12.8 | 13.8 | 70.9 | 30.1 | 4.2 |
| P0003Z-03 | 194 | 552.5 | 7.8 | 15.6 | 119.7 | 42.3 | 8.6 |
| P0002L-03 | 2790 | 271.8 | 6.9 | 14.3 | 189.4 | 69.7 | 7.2 |
| P00057-03 | 1622 | 6918.9 | 70.2 | 16.9 | 231 | 43.3 | 5.2 |
| P00076-0l | 152 | 987.7 | 21.2 | 8.4 | 168.9 | 63 | 16.3 |
| P00077-0l | 1558 | 8888.5 | 102.2 | 9.7 | 119.6 | 80.2 | 5.4 |
| P0002M-04 | 7200 | 1330.6 | 13.7 | 20.6 | 210.9 | 95.5 | 12.5 |
| P0007C-0l | 271 | 526.6 | 4.7 | 12.8 | 115.1 | 66.8 | 7.1 |
| P00079-0l | 585 | 513 | 7.5 | 16.5 | 171.9 | 79.3 | 7.4 |
| P0007D-0l | 5495 | 302.5 | 10.5 | 9.4 | 149.4 | 51.2 | 18.7 |
| P0002F-02 | 7663 | 670.4 | 236.8 | 11 | 136.2 | 90.8 | 14 |
| P0007E-0l | 11836 | 1856.1 | 12.8 | 1.3 | 63.6 | 27.4 | 17.4 |
| P0007F-0l | 491 | 2179.6 | 34.1 | 95.2 | 587.5 | 161.2 | 90.5 |
| P0007G-0l | 108 | 3771.5 | 25.2 | 26 | 141.7 | 51.5 | 18.1 |
| P0002A-03 | 5218 | 1214.1 | 21.2 | 87.9 | 443.2 | 78.3 | 93.8 |
| P0005U-03 | 550 | 10511 | 160 | 38.9 | 253.5 | 88.4 | 77 |
| P0007H-0l | 105 | 5633.6 | 72.1 | 7.6 | 222.1 | 51 | 13.6 |
| P00071-0l | 317 | 133.4 | 25.1 | 6.5 | 227.3 | 110.9 | 11.2 |
| P00068-02 | 1070 | 185.8 | 2.6 | 1.7 | 42.2 | 31.9 | 4.5 |
| P0006D-0l | 112 | 4020.6 | 35.9 | 198.9 | 1917.2 | 99.3 | 1117 |
| P0005W-02 | 108 | 2044.5 | 19.3 | 31.6 | 187.5 | 67.4 | 23.7 |
| P0003M-02 | 2714 | 1301.1 | 1656.1 | 11.9 | 67.2 | 36.6 | 12.2 |
| P0004Y-02 | 2292 | 29.8 | 7.9 | 37.8 | 271.8 | 83.2 | 11.1 |
| P0004X-0l | 382 | 109.8 | 21.2 | 19.7 | 506 | 64.9 | 95.7 |
| P00056-0l | 3711 | 177.6 | 5 | 10.5 | 117.4 | 83.8 | 29.7 |
| P00057-0l | 1517 | 2090.4 | 101.3 | 8 | 66.6 | 20 | 2.5 |
| P00058-0l | 224 | 479.6 | 122 | 55.3 | 530.5 | 127.7 | 13 |
| P0002T-02 | 2012 | 622.8 | 35.1 | 50.6 | 881.8 | 124.4 | 41.8 |
| P00059-0l | 56 | 18.5 | 4.5 | 2.8 | 50.4 | 31.3 | 2.4 |
| P0000P-02 | 5184 | 479.5 | 46.5 | 22.7 | 349.4 | 159.7 | 93.7 |
| P0005A-0l | 1554 | 115.6 | 27.6 | 27.8 | 631.1 | 196.8 | 41.2 |
| P0005B-0l | 1043 | 1539.3 | 18.9 | 15.3 | 221.3 | 160.4 | 10.2 |
| P0000J-03 | 371 | 5.2 | 5.2 | 1.5 | 50.7 | 17.9 | 2.6 |
| P0005C-0l | 53 | 3201.7 | 47 | 21.4 | 194.8 | 33.5 | 25.2 |
| P0002l-02 | 10209 | 1112.1 | 167.6 | 55.1 | 1886.1 | 46.9 | 252.4 |
| P0003O-02 | 1332 | 14.1 | 10.6 | 21.2 | 139.7 | 99.5 | 13.4 |
| P0000A-03 | 812 | 186.4 | 4.4 | 11.2 | 87.2 | 78.8 | 7.3 |
| P0004Y-03 | 2310 | 21.7 | 5.5 | 23.3 | 174.7 | 53.1 | 5 |
| P000lU-04 | 6403 | 183.1 | 15.1 | 9.3 | 127.9 | 76.9 | 25.1 |
| P000lK-02 | 254 | 1295 | 57.4 | 20 | 354.8 | 44.1 | 1.8 |
| P0002H-02 | 3145 | 33.2 | 19.4 | 5.6 | 70 | 48.7 | 8.6 |
| P00027-02 | 2432 | 1048 | 302.7 | 520.2 | 535.5 | 48.7 | 58.2 |
| P0002D-02 | 4175 | 2147.9 | 191.3 | 24.9 | 303.1 | 177.1 | 13.9 |

An analyte tolerance calculator was used to stratify the performance of six individual biomarkers detected in QSant™. Briefly, the input of six different biomarkers was considered in the context of the QSant™ algorithm as used in the AQUA registry study. The algorithm includes normalizations to creatine and other transformations to substantially account for changes in the biomarker levels observed when samples were shipped from one location to another. The algorithm was used to provide the Q-Score. TABLE 2A illustrates a relationship between the Q-Score and the threshold values for the rapid flow device. Boolean logic for column TABLE 2A is as follows: if either m-cIDNA is its threshold m-cIDNA (25 ng/mL) and total protein (25 ng/mL), or total protein is its threshold (25 ng/ml), then column Lis TRUE, otherwise column L is FALSE.

TABLE 2A

| | Q- | mcIDNA High- | One or more protein(s) | | Q- |
|---|---|---|---|---|---|
| ID | Score | Low | High-Low | OR | SCORE |
| P0002N-03 | 37 | FALSE | FALSE | FALSE | REJECT |
| P00071-01 | 53 | TRUE | FALSE | TRUE | REJECT |
| P0004W-01 | 13 | FALSE | FALSE | FALSE | OK |
| P0003W-02 | 14 | FALSE | TRUE | TRUE | OK |
| P0005R-01 | 52 | TRUE | TRUE | TRUE | REJECT |
| P0004O-02 | 9 | FALSE | FALSE | FALSE | OK |
| P00046-02 | 27 | FALSE | FALSE | FALSE | OK |
| P0005M-01 | 65 | TRUE | FALSE | TRUE | REJECT |
| P00058-02 | 58 | TRUE | FALSE | TRUE | REJECT |
| P00061-01 | 27 | FALSE | TRUE | TRUE | OK |
| P00060-01 | 17 | FALSE | FALSE | FALSE | OK |
| P0000U-02 | 82 | TRUE | FALSE | TRUE | REJECT |
| P000lU-05 | 41 | FALSE | TRUE | TRUE | REJECT |
| P00036-02 | 19 | FALSE | FALSE | FALSE | OK |
| P0003X-02 | 13 | FALSE | FALSE | FALSE | OK |
| P00066-01 | 35 | TRUE | TRUE | TRUE | REJECT |
| P00065-01 | 17 | FALSE | FALSE | FALSE | OK |
| P00067-01 | 17 | TRUE | TRUE | TRUE | OK |
| P00069-01 | 48 | TRUE | TRUE | TRUE | REJECT |
| P0006A-01 | 83 | TRUE | TRUE | TRUE | REJECT |
| P00027-03 | 90 | TRUE | TRUE | TRUE | REJECT |
| P0006B-01 | 35 | TRUE | FALSE | TRUE | REJECT |
| P0006C-01 | 36 | TRUE | TRUE | TRUE | REJECT |
| P0002V-02 | 62 | TRUE | FALSE | TRUE | REJECT |
| P0003S-02 | 13 | FALSE | FALSE | FALSE | OK |
| P00062-01 | 91 | TRUE | TRUE | TRUE | REJECT |
| P0004O-03 | 11 | FALSE | FALSE | FALSE | OK |
| P0005H-02 | 50 | TRUE | FALSE | TRUE | REJECT |
| P0003l-02 | 13 | FALSE | FALSE | FALSE | OK |
| P0005P-01 | 66 | FALSE | TRUE | TRUE | REJECT |
| P0005N-01 | 28 | FALSE | TRUE | TRUE | OK |
| P0005O-01 | 98 | TRUE | TRUE | TRUE | REJECT |
| P0005T-01 | 96 | TRUE | TRUE | TRUE | REJECT |
| P0005V-01 | 59 | TRUE | TRUE | TRUE | REJECT |
| P0004J-02 | 32 | FALSE | TRUE | TRUE | OK |
| P0005U-01 | 49 | TRUE | FALSE | TRUE | REJECT |
| P000lQ-02 | 36 | FALSE | FALSE | FALSE | REJECT |
| P000lG-02 | 83 | TRUE | TRUE | TRUE | REJECT |
| P0005Y-01 | 36 | TRUE | TRUE | TRUE | REJECT |
| P0005W-01 | 61 | TRUE | FALSE | TRUE | REJECT |
| P0005X-01 | 83 | TRUE | TRUE | TRUE | REJECT |
| P0002N-02 | 36 | FALSE | TRUE | TRUE | REJECT |
| P0005Z-01 | 80 | TRUE | TRUE | TRUE | REJECT |
| P0005H-01 | 34 | FALSE | FALSE | FALSE | REJECT |
| P00008-03 | 55 | TRUE | FALSE | TRUE | REJECT |
| P0003Q-05 | 28 | TRUE | FALSE | TRUE | OK |
| P0002C-02 | 22 | FALSE | FALSE | FALSE | OK |
| P00026-02 | 13 | FALSE | FALSE | FALSE | OK |
| P0003Q-03 | 88 | TRUE | FALSE | TRUE | REJECT |
| P0005J-01 | 80 | TRUE | FALSE | TRUE | REJECT |
| P0002B-02 | 58 | TRUE | TRUE | TRUE | REJECT |
| P0005I-01 | 35 | TRUE | FALSE | TRUE | REJECT |
| P0002J-03 | 25 | FALSE | TRUE | TRUE | OK |
| P0005K-01 | 36 | FALSE | FALSE | FALSE | REJECT |
| P000lS-02 | 31 | FALSE | TRUE | TRUE | OK |
| P0004F-02 | 67 | TRUE | TRUE | TRUE | REJECT |
| P00063-01 | 16 | FALSE | TRUE | TRUE | OK |
| P00052-02 | 30 | FALSE | FALSE | FALSE | OK |
| P00064-01 | 36 | TRUE | FALSE | TRUE | REJECT |
| P0007J-01 | 18 | FALSE | FALSE | FALSE | OK |
| P0005T-02 | 86 | TRUE | TRUE | TRUE | REJECT |
| P0007K-01 | 30 | FALSE | FALSE | FALSE | OK |
| P0007L-01 | 36 | FALSE | FALSE | FALSE | REJECT |
| P0007M-01 | 49 | TRUE | FALSE | TRUE | REJECT |
| P0006E-02 | 10 | FALSE | TRUE | TRUE | OK |
| P00021-01 | 91 | TRUE | FALSE | TRUE | REJECT |
| P00059-02 | 10 | FALSE | FALSE | FALSE | OK |
| P0007O-01 | 31 | FALSE | FALSE | FALSE | OK |
| P0002V-04 | 41 | TRUE | FALSE | TRUE | REJECT |
| P0007P-01 | 39 | FALSE | TRUE | TRUE | REJECT |
| P0007Q-01 | 41 | TRUE | TRUE | TRUE | REJECT |
| P0007R-01 | 60 | FALSE | TRUE | TRUE | REJECT |
| P0007T-01 | 36 | FALSE | FALSE | FALSE | REJECT |
| P0007S-01 | 30 | FALSE | FALSE | FALSE | OK |
| P00004-01 | 53 | TRUE | TRUE | TRUE | REJECT |
| P00005-01 | 27 | FALSE | FALSE | FALSE | OK |
| P00007-01 | 51 | TRUE | TRUE | TRUE | REJECT |
| P00006-01 | 42 | FALSE | TRUE | TRUE | REJECT |
| P00008-01 | 19 | FALSE | FALSE | FALSE | OK |
| P0000D-01 | 39 | TRUE | TRUE | TRUE | REJECT |
| P0000B-01 | 66 | TRUE | FALSE | TRUE | REJECT |
| P0000C-01 | 45 | TRUE | TRUE | TRUE | REJECT |
| P0000A-01 | 25 | FALSE | FALSE | FALSE | OK |
| P0000F-01 | 13 | FALSE | FALSE | FALSE | OK |
| P0000E-01 | 36 | FALSE | TRUE | TRUE | REJECT |
| P00002-01 | 39 | TRUE | TRUE | TRUE | REJECT |
| P00003-01 | 13 | TRUE | TRUE | TRUE | OK |
| P0000P-01 | 53 | TRUE | TRUE | TRUE | REJECT |
| P0000O-01 | 30 | FALSE | FALSE | FALSE | OK |
| P0000Q-01 | 15 | TRUE | TRUE | TRUE | OK |
| P0000R-01 | 36 | FALSE | FALSE | FALSE | REJECT |
| P0000S-01 | 36 | TRUE | FALSE | TRUE | REJECT |
| P0000U-01 | 83 | TRUE | FALSE | TRUE | REJECT |
| P00004-02 | 33 | TRUE | TRUE | TRUE | REJECT |
| P0000W-01 | 36 | FALSE | FALSE | FALSE | REJECT |
| P0000X-01 | 41 | FALSE | TRUE | TRUE | REJECT |
| P0000V-01 | 36 | FALSE | TRUE | TRUE | REJECT |
| P0000Y-01 | 75 | TRUE | FALSE | TRUE | REJECT |
| P0000Z-01 | 50 | TRUE | FALSE | TRUE | REJECT |
| P0000B-02 | 41 | TRUE | TRUE | TRUE | REJECT |
| P0000C-02 | 43 | FALSE | TRUE | TRUE | REJECT |
| P0000M-02 | 30 | TRUE | FALSE | TRUE | OK |
| P00012-01 | 41 | FALSE | FALSE | FALSE | REJECT |
| P000ll-01 | 31 | FALSE | FALSE | FALSE | OK |
| P000l0-01 | 37 | FALSE | FALSE | FALSE | REJECT |
| P0001V-02 | 52 | TRUE | FALSE | TRUE | REJECT |
| P00013-01 | 45 | FALSE | FALSE | FALSE | REJECT |
| P000lV-03 | 52 | TRUE | FALSE | TRUE | REJECT |
| P00016-02 | 13 | FALSE | FALSE | FALSE | OK |
| P000lA-01 | 83 | TRUE | FALSE | TRUE | REJECT |
| P000l7-01 | 35 | TRUE | FALSE | TRUE | REJECT |
| P00019-01 | 20 | FALSE | FALSE | FALSE | OK |
| P00015-01 | 10 | FALSE | FALSE | FALSE | OK |
| P00018-01 | 77 | TRUE | FALSE | TRUE | REJECT |
| P00014-01 | 39 | FALSE | FALSE | FALSE | REJECT |
| P000lC-01 | 20 | FALSE | FALSE | FALSE | OK |
| P000lB-01 | 83 | TRUE | FALSE | TRUE | REJECT |
| P000lD-01 | 30 | FALSE | FALSE | FALSE | OK |
| P000lV-01 | 52 | TRUE | FALSE | TRUE | REJECT |
| P0000H-01 | 27 | FALSE | FALSE | FALSE | OK |
| P00009-02 | 50 | TRUE | FALSE | TRUE | REJECT |
| P0000G-01 | 38 | TRUE | TRUE | TRUE | REJECT |
| P000lV-06 | 91 | TRUE | FALSE | TRUE | REJECT |
| P0000K-01 | 27 | FALSE | TRUE | TRUE | OK |
| P000OI-01 | 41 | TRUE | TRUE | TRUE | REJECT |
| PO000J-01 | 13 | FALSE | FALSE | FALSE | OK |
| P0000L-01 | 41 | TRUE | FALSE | TRUE | REJECT |
| P0000M-01 | 82 | TRUE | FALSE | TRUE | REJECT |
| P0000N-01 | 43 | TRUE | TRUE | TRUE | REJECT |
| P000IJ-01 | 68 | TRUE | TRUE | TRUE | REJECT |
| P00005-02 | 19 | FALSE | FALSE | FALSE | OK |
| P0000O-02 | 13 | FALSE | TRUE | TRUE | OK |
| P000lG-01 | 83 | TRUE | TRUE | TRUE | REJECT |
| P000lH-01 | 68 | TRUE | TRUE | TRUE | REJECT |
| P000II-01 | 75 | TRUE | TRUE | TRUE | REJECT |
| P000lV-13 | 43 | TRUE | TRUE | TRUE | REJECT |
| P000lM-02 | 25 | FALSE | FALSE | FALSE | OK |
| P000lP-01 | 10 | FALSE | FALSE | FALSE | OK |
| P000lQ-01 | 34 | TRUE | TRUE | TRUE | REJECT |
| P000lV-08 | 59 | TRUE | FALSE | TRUE | REJECT |
| P000lS-01 | 53 | TRUE | FALSE | TRUE | REJECT |
| P000lL-03 | 13 | FALSE | FALSE | FALSE | OK |
| P000lR-01 | 49 | TRUE | FALSE | TRUE | REJECT |
| P000lO-02 | 75 | TRUE | TRUE | TRUE | REJECT |
| P000lU-01 | 41 | FALSE | TRUE | TRUE | REJECT |
| P000lZ-01 | 83 | TRUE | TRUE | TRUE | REJECT |
| P0000T-02 | 57 | TRUE | FALSE | TRUE | REJECT |
| P000lT-01 | 13 | FALSE | TRUE | TRUE | OK |
| P0001X-01 | 31 | TRUE | FALSE | TRUE | OK |
| P000lY-01 | 54 | TRUE | TRUE | TRUE | REJECT |
| P0000Z-02 | 27 | FALSE | FALSE | FALSE | OK |
| P000lW-01 | 27 | FALSE | FALSE | FALSE | OK |
| P000lV-09 | 59 | TRUE | FALSE | TRUE | REJECT |
| P00020-01 | 13 | FALSE | FALSE | FALSE | OK |
| P000lN-01 | 40 | TRUE | TRUE | TRUE | REJECT |
| P000lO-01 | 53 | FALSE | FALSE | FALSE | REJECT |
| P0001V-12 | 41 | TRUE | FALSE | TRUE | REJECT |
| P00022-01 | 50 | TRUE | TRUE | TRUE | REJECT |
| P00024-01 | 31 | FALSE | FALSE | FALSE | OK |
| P00025-01 | 36 | TRUE | TRUE | TRUE | REJECT |
| P00028-01 | 34 | FALSE | TRUE | TRUE | REJECT |
| P00026-01 | 23 | FALSE | FALSE | FALSE | OK |
| P00027-01 | 31 | FALSE | FALSE | FALSE | OK |
| P0002D-01 | 82 | TRUE | FALSE | TRUE | REJECT |
| P0002G-01 | 24 | FALSE | FALSE | FALSE | OK |
| P0000Z-03 | 39 | TRUE | TRUE | TRUE | REJECT |
| P0002B-01 | 40 | FALSE | FALSE | FALSE | REJECT |
| P0002A-01 | 34 | FALSE | TRUE | TRUE | REJECT |
| P0002E-01 | 22 | FALSE | FALSE | FALSE | OK |
| P0002F-01 | 16 | FALSE | FALSE | FALSE | OK |
| P0002C-01 | 35 | FALSE | FALSE | FALSE | REJECT |
| P0000T-03 | 40 | TRUE | FALSE | TRUE | REJECT |
| P0002O-01 | 41 | TRUE | TRUE | TRUE | REJECT |
| P0000G-02 | 59 | TRUE | FALSE | TRUE | REJECT |
| P0002I-01 | 51 | TRUE | TRUE | TRUE | REJECT |
| P0002K-01 | 43 | FALSE | FALSE | FALSE | REJECT |
| P0002H-01 | 27 | FALSE | FALSE | FALSE | OK |
| P0002J-01 | 12 | FALSE | FALSE | FALSE | OK |
| P0002L-01 | 13 | FALSE | FALSE | FALSE | OK |
| P0002M-01 | 36 | TRUE | FALSE | TRUE | REJECT |
| P0000Z-04 | 22 | FALSE | FALSE | FALSE | OK |
| P0002N-01 | 13 | FALSE | FALSE | FALSE | OK |
| P0002R-01 | 60 | TRUE | FALSE | TRUE | REJECT |
| P0002P-01 | 46 | TRUE | FALSE | TRUE | REJECT |
| P0002G-02 | 27 | FALSE | FALSE | FALSE | OK |
| P0002S-01 | 78 | TRUE | TRUE | TRUE | REJECT |
| P0002T-01 | 36 | FALSE | FALSE | FALSE | REJECT |
| P0002U-01 | 13 | FALSE | FALSE | FALSE | OK |
| P0002V-01 | 58 | TRUE | TRUE | TRUE | REJECT |
| P00023-01 | 34 | FALSE | TRUE | TRUE | REJECT |
| P000lU-02 | 15 | FALSE | TRUE | TRUE | OK |
| P00033-01 | 37 | FALSE | TRUE | TRUE | REJECT |
| P00007-02 | 45 | FALSE | TRUE | TRUE | REJECT |
| P00029-02 | 22 | FALSE | TRUE | TRUE | OK |
| P00034-01 | 43 | TRUE | TRUE | TRUE | REJECT |
| P00035-01 | 10 | FALSE | FALSE | FALSE | OK |
| P0000B-03 | 38 | TRUE | FALSE | TRUE | REJECT |
| P00037-01 | 12 | FALSE | TRUE | TRUE | OK |
| P00036-01 | 10 | FALSE | FALSE | FALSE | OK |
| P00038-01 | 13 | FALSE | TRUE | TRUE | OK |
| P0003B-01 | 83 | TRUE | FALSE | TRUE | REJECT |
| P0003A-01 | 13 | TRUE | FALSE | TRUE | OK |
| P0003D-01 | 51 | TRUE | TRUE | TRUE | REJECT |
| P0003C-01 | 36 | FALSE | TRUE | TRUE | REJECT |
| P0003E-01 | 31 | FALSE | FALSE | FALSE | OK |
| P00039-02 | 38 | TRUE | TRUE | TRUE | REJECT |
| P0002X-02 | 37 | TRUE | FALSE | TRUE | REJECT |
| P0003F-01 | 44 | TRUE | TRUE | TRUE | REJECT |
| P00009-03 | 29 | FALSE | TRUE | TRUE | OK |
| P0002Z-01 | 31 | FALSE | TRUE | TRUE | OK |
| P0002W-01 | 13 | FALSE | TRUE | TRUE | OK |
| P0003I-01 | 33 | FALSE | FALSE | FALSE | REJECT |
| P0003H-01 | 66 | TRUE | FALSE | TRUE | REJECT |
| P000lZ-02 | 69 | TRUE | TRUE | TRUE | REJECT |
| P0002Y-01 | 72 | TRUE | TRUE | TRUE | REJECT |
| P00030-01 | 25 | FALSE | FALSE | FALSE | OK |
| P000lJ-02 | 37 | TRUE | TRUE | TRUE | REJECT |
| P0003N-01 | 44 | TRUE | FALSE | TRUE | REJECT |
| P0003J-01 | 25 | FALSE | FALSE | FALSE | OK |
| P0003G-02 | 46 | TRUE | FALSE | TRUE | REJECT |
| P0003K-01 | 32 | FALSE | FALSE | FALSE | OK |
| P0003M-01 | 40 | TRUE | FALSE | TRUE | REJECT |
| P0002Q-02 | 18 | TRUE | FALSE | TRUE | OK |
| P00032-01 | 10 | FALSE | FALSE | FALSE | OK |
| P0003l-01 | 22 | FALSE | FALSE | FALSE | OK |
| P0006H-01 | 13 | FALSE | FALSE | FALSE | OK |
| P0006F-01 | 13 | FALSE | FALSE | FALSE | OK |
| P00057-02 | 83 | TRUE | FALSE | TRUE | REJECT |
| P0006I-01 | 27 | FALSE | FALSE | FALSE | OK |
| P0006K-01 | 58 | TRUE | TRUE | TRUE | REJECT |
| P0006E-01 | 34 | TRUE | TRUE | TRUE | REJECT |
| P0006G-01 | 36 | FALSE | FALSE | FALSE | REJECT |
| P0006L-01 | 38 | FALSE | FALSE | FALSE | REJECT |
| P0002V-03 | 83 | TRUE | FALSE | TRUE | REJECT |
| P0006M-01 | 68 | TRUE | FALSE | TRUE | REJECT |
| P0006N-01 | 25 | FALSE | FALSE | FALSE | OK |
| P0006O-01 | 45 | TRUE | TRUE | TRUE | REJECT |
| P0002B-03 | 83 | TRUE | TRUE | TRUE | REJECT |
| P0006R-01 | 56 | TRUE | FALSE | TRUE | REJECT |
| P0006X-01 | 31 | FALSE | FALSE | FALSE | OK |
| P0006Q-01 | 49 | TRUE | FALSE | TRUE | REJECT |
| P0006S-01 | 25 | FALSE | TRUE | TRUE | OK |
| P0006W-01 | 13 | FALSE | FALSE | FALSE | OK |
| P0006Y-01 | 22 | FALSE | TRUE | TRUE | OK |
| P0006T-01 | 34 | FALSE | FALSE | FALSE | REJECT |
| P0006V-01 | 22 | FALSE | FALSE | FALSE | OK |
| P0007B-01 | 60 | TRUE | TRUE | TRUE | REJECT |
| P0007A-01 | 49 | TRUE | TRUE | TRUE | REJECT |
| P0006C-02 | 36 | FALSE | TRUE | TRUE | REJECT |
| P0006Z-01 | 36 | TRUE | FALSE | TRUE | REJECT |
| P00072-01 | 25 | TRUE | TRUE | TRUE | OK |
| P0006P-02 | 19 | FALSE | FALSE | FALSE | OK |
| P0003P-01 | 40 | TRUE | FALSE | TRUE | REJECT |
| P00009-04 | 16 | TRUE | TRUE | TRUE | OK |
| P0000M-03 | 52 | TRUE | FALSE | TRUE | REJECT |
| P000l2-02 | 22 | TRUE | FALSE | TRUE | OK |
| P0003R-01 | 13 | FALSE | FALSE | FALSE | OK |
| P0003X-01 | 52 | FALSE | FALSE | FALSE | REJECT |
| P0003S-01 | 15 | FALSE | FALSE | FALSE | OK |
| P0003T-01 | 13 | FALSE | FALSE | FALSE | OK |
| P0003W-01 | 13 | TRUE | TRUE | TRUE | OK |
| P0003U-01 | 68 | TRUE | FALSE | TRUE | REJECT |
| P0003Y-01 | 47 | FALSE | TRUE | TRUE | REJECT |
| P0003V-01 | 42 | TRUE | TRUE | TRUE | REJECT |
| P0000C-03 | 50 | TRUE | TRUE | TRUE | REJECT |
| P0000H-02 | 40 | TRUE | FALSE | TRUE | REJECT |
| P0000O-03 | 22 | FALSE | FALSE | FALSE | OK |
| P00040-01 | 36 | FALSE | TRUE | TRUE | REJECT |
| P0003Z-01 | 14 | TRUE | FALSE | TRUE | OK |
| P0003N-02 | 37 | TRUE | FALSE | TRUE | REJECT |
| P00042-01 | 41 | TRUE | FALSE | TRUE | REJECT |
| P00041-01 | 40 | TRUE | FALSE | TRUE | REJECT |
| P0000J-02 | 13 | FALSE | FALSE | FALSE | OK |
| P00043-01 | 43 | TRUE | FALSE | TRUE | REJECT |
| P00044-01 | 45 | TRUE | FALSE | TRUE | REJECT |
| P0000L-02 | 13 | FALSE | FALSE | FALSE | OK |
| P000ll-02 | 13 | FALSE | FALSE | FALSE | OK |
| P0003Q-02 | 33 | TRUE | TRUE | TRUE | REJECT |
| P0002M-02 | 44 | TRUE | FALSE | TRUE | REJECT |
| P00049-01 | 34 | TRUE | FALSE | TRUE | REJECT |
| P00070-01 | 81 | TRUE | FALSE | TRUE | REJECT |
| P0005J-02 | 35 | FALSE | TRUE | TRUE | REJECT |
| P0006J-02 | 36 | FALSE | FALSE | FALSE | REJECT |
| P0006U-02 | 27 | FALSE | FALSE | FALSE | OK |
| P00073-01 | 25 | TRUE | FALSE | TRUE | OK |
| P00075-01 | 23 | TRUE | TRUE | TRUE | OK |
| P00078-01 | 19 | FALSE | FALSE | FALSE | OK |
| P0003Z-03 | 13 | FALSE | FALSE | FALSE | OK |
| P0002L-03 | 36 | FALSE | FALSE | FALSE | REJECT |
| P00057-03 | 83 | TRUE | FALSE | TRUE | REJECT |
| P00076-01 | 17 | FALSE | FALSE | FALSE | OK |
| P00077-01 | 68 | TRUE | FALSE | TRUE | REJECT |
| P0002M-04 | 36 | FALSE | FALSE | FALSE | REJECT |
| P0007C-01 | 41 | FALSE | FALSE | FALSE | REJECT |
| P00079-01 | 36 | FALSE | FALSE | FALSE | REJECT |
| P0007D-01 | 27 | FALSE | FALSE | FALSE | OK |
| P0002F-02 | 47 | TRUE | FALSE | TRUE | REJECT |
| P0007E-01 | 29 | FALSE | FALSE | FALSE | OK |
| P0007F-01 | 38 | TRUE | TRUE | TRUE | REJECT |
| P0007G-01 | 34 | TRUE | FALSE | TRUE | REJECT |
| P0002A-03 | 36 | FALSE | TRUE | TRUE | REJECT |
| P0005U-03 | 68 | TRUE | TRUE | TRUE | REJECT |
| P0007H-01 | 67 | TRUE | FALSE | TRUE | REJECT |
| P0007l-01 | 53 | TRUE | FALSE | TRUE | REJECT |
| P00068-02 | 13 | FALSE | FALSE | FALSE | OK |
| P0006D-01 | 60 | TRUE | TRUE | TRUE | REJECT |
| P0005W-02 | 24 | FALSE | FALSE | FALSE | OK |
| P0003M-02 | 26 | TRUE | FALSE | TRUE | OK |
| P0004Y-02 | 36 | FALSE | FALSE | FALSE | REJECT |
| P0004X-01 | 36 | FALSE | TRUE | TRUE | REJECT |
| P00056-01 | 34 | FALSE | TRUE | TRUE | REJECT |
| P00057-01 | 53 | TRUE | FALSE | TRUE | REJECT |
| P00058-01 | 36 | TRUE | FALSE | TRUE | REJECT |
| P0002T-02 | 43 | TRUE | TRUE | TRUE | REJECT |
| P00059-01 | 12 | FALSE | FALSE | FALSE | OK |
| P0000P-02 | 46 | TRUE | TRUE | TRUE | REJECT |
| P0005A-01 | 36 | TRUE | TRUE | TRUE | REJECT |
| P0005B-01 | 34 | FALSE | FALSE | FALSE | REJECT |
| P0000J-03 | 14 | FALSE | FALSE | FALSE | OK |
| P0005C-01 | 56 | TRUE | TRUE | TRUE | REJECT |
| P0002I-02 | 68 | TRUE | TRUE | TRUE | REJECT |
| P0003O-02 | 36 | FALSE | FALSE | FALSE | REJECT |
| P0000A-03 | 30 | FALSE | FALSE | FALSE | OK |
| P0004Y-03 | 25 | FALSE | FALSE | FALSE | OK |
| P000lU-04 | 34 | FALSE | TRUE | TRUE | REJECT |
| P000lK-02 | 41 | TRUE | FALSE | TRUE | REJECT |
| P0002H-02 | 26 | FALSE | FALSE | FALSE | OK |
| P00027-02 | 62 | TRUE | TRUE | TRUE | REJECT |
| P0002D-02 | 41 | TRUE | FALSE | TRUE | REJECT |

The results were further stratified as follows:

IF Boolean logic column “OR” is TRUE and column Q-Score is “REJECT”, column one or more protein(s) HighLow is TRUE, column methyl_cIDNA HighLow is TRUE. This represents a true positive.

IF Boolean logic column “OR” is FALSE and column Q-Score is “OK”, column one or more protein(s) HighLow is FALSE, column methyl_cIDNA HighLow is FALSE. This represents a true negative.

Boolean logic column “OR” is TRUE and column Q-Score is “OK”, column one or more protein(s) HighLow is TRUE, column methyl_cIDNA HighLow is FALSE. This represents a false positive.

IF Boolean logic column “OR” is FALSE and column Q-Score is “REJECT”, column one or more protein(s) HighLow is FALSE, column methyl_cIDNA HighLow is TRUE. This represents a false negative.

TABLE 2B provides a more succinct tabulation of the individual results presented in

TABLE 2B

| | OK | REJECT | |
|---|---|---|---|
| OK | 87 | 36 | 123 |
| REJECT | 30 | 176 | 206 |
| Total | 117 | 212 | 329 |

The calculation for sensitivity was performed as follows: TP/(TP+FN), where TP is the total of true positives and FN is the total of false negatives; this fraction was expressed as a percentage. The calculation for specificity is TN/(TN+FP), where TN is the total of true negatives and FP is the total of false positives; this fraction was also expressed as a percentage. TABLE 3A provides a summary of true positive (TP), true negative (TN), false positive (FP), and false negative (FN) detection of rejection by the lateral flow assay (LFA) using the cutoff values of 25 ng/ml of m-cIDNA and 25 ng/ml of total protein, respectively, which the device should achieve to perform as intended.

TABLE 3A

| ID | TP | TN | FP | FN |
|---|---|---|---|---|
| P0002N-03 | FALSE | FALSE | FALSE | TRUE |
| P00071-01 | TRUE | FALSE | FALSE | FALSE |
| P0004W-01 | FALSE | TRUE | FALSE | FALSE |
| P0003W-02 | FALSE | FALSE | TRUE | FALSE |
| P0005R-01 | TRUE | FALSE | FALSE | FALSE |
| P0004O-02 | FALSE | TRUE | FALSE | FALSE |
| P00046-02 | FALSE | TRUE | FALSE | FALSE |
| P0005M-01 | TRUE | FALSE | FALSE | FALSE |
| P00058-02 | TRUE | FALSE | FALSE | FALSE |
| P00061-01 | FALSE | FALSE | TRUE | FALSE |
| P00060-01 | FALSE | TRUE | FALSE | FALSE |
| P0000U-02 | TRUE | FALSE | FALSE | FALSE |
| P000lU-05 | TRUE | FALSE | FALSE | FALSE |
| P00036-02 | FALSE | TRUE | FALSE | FALSE |
| P0003X-02 | FALSE | TRUE | FALSE | FALSE |
| P00066-01 | TRUE | FALSE | FALSE | FALSE |
| P00065-01 | FALSE | TRUE | FALSE | FALSE |
| P00067-01 | FALSE | FALSE | TRUE | FALSE |
| P00069-01 | TRUE | FALSE | FALSE | FALSE |
| P0006A-01 | TRUE | FALSE | FALSE | FALSE |
| P00027-03 | TRUE | FALSE | FALSE | FALSE |
| P0006B-01 | TRUE | FALSE | FALSE | FALSE |
| P0006C-01 | TRUE | FALSE | FALSE | FALSE |
| P0002V-02 | TRUE | FALSE | FALSE | FALSE |
| P0003S-02 | FALSE | TRUE | FALSE | FALSE |
| P00062-01 | TRUE | FALSE | FALSE | FALSE |
| P0004O-03 | FALSE | TRUE | FALSE | FALSE |
| P0005H-02 | TRUE | FALSE | FALSE | FALSE |
| P0003l-02 | FALSE | TRUE | FALSE | FALSE |
| P0005P-01 | TRUE | FALSE | FALSE | FALSE |
| P0005N-01 | FALSE | FALSE | TRUE | FALSE |
| P0005O-01 | TRUE | FALSE | FALSE | FALSE |
| P0005T-01 | TRUE | FALSE | FALSE | FALSE |
| P0005V-01 | TRUE | FALSE | FALSE | FALSE |
| P0004J-02 | FALSE | FALSE | TRUE | FALSE |
| P0005U-01 | TRUE | FALSE | FALSE | FALSE |
| P000lQ-02 | FALSE | FALSE | FALSE | TRUE |
| P000lG-02 | TRUE | FALSE | FALSE | FALSE |
| P0005Y-01 | TRUE | FALSE | FALSE | FALSE |
| P0005W-01 | TRUE | FALSE | FALSE | FALSE |
| P0005X-01 | TRUE | FALSE | FALSE | FALSE |
| P0002N-02 | TRUE | FALSE | FALSE | FALSE |
| P0005Z-01 | TRUE | FALSE | FALSE | FALSE |
| P0005H-01 | FALSE | FALSE | FALSE | TRUE |
| P00008-03 | TRUE | FALSE | FALSE | FALSE |
| P0003Q-05 | FALSE | FALSE | TRUE | FALSE |
| P0002C-02 | FALSE | TRUE | FALSE | FALSE |
| P00026-02 | FALSE | TRUE | FALSE | FALSE |
| P0003Q-03 | TRUE | FALSE | FALSE | FALSE |
| P0005J-01 | TRUE | FALSE | FALSE | FALSE |
| P0002B-02 | TRUE | FALSE | FALSE | FALSE |
| P0005I-01 | TRUE | FALSE | FALSE | FALSE |
| P0002J-03 | FALSE | FALSE | TRUE | FALSE |
| P0005K-01 | FALSE | FALSE | FALSE | TRUE |
| P000lS-02 | FALSE | FALSE | TRUE | FALSE |
| P0004F-02 | TRUE | FALSE | FALSE | FALSE |
| P00063-01 | FALSE | FALSE | TRUE | FALSE |
| P00052-02 | FALSE | TRUE | FALSE | FALSE |
| P00064-01 | TRUE | FALSE | FALSE | FALSE |
| P0007J-01 | FALSE | TRUE | FALSE | FALSE |
| P0005T-02 | TRUE | FALSE | FALSE | FALSE |
| P0007K-01 | FALSE | TRUE | FALSE | FALSE |
| P0007L-01 | FALSE | FALSE | FALSE | TRUE |
| P0007M-01 | TRUE | FALSE | FALSE | FALSE |
| P0006E-02 | FALSE | FALSE | TRUE | FALSE |
| P00021-01 | TRUE | FALSE | FALSE | FALSE |
| P00059-02 | FALSE | TRUE | FALSE | FALSE |
| P0007O-01 | FALSE | TRUE | FALSE | FALSE |
| P0002V-04 | TRUE | FALSE | FALSE | FALSE |
| P0007P-01 | TRUE | FALSE | FALSE | FALSE |
| P0007Q-01 | TRUE | FALSE | FALSE | FALSE |
| P0007R-01 | TRUE | FALSE | FALSE | FALSE |
| P0007T-01 | FALSE | FALSE | FALSE | TRUE |
| P0007S-01 | FALSE | TRUE | FALSE | FALSE |
| P00004-01 | TRUE | FALSE | FALSE | FALSE |
| P00005-01 | FALSE | TRUE | FALSE | FALSE |
| P00007-01 | TRUE | FALSE | FALSE | FALSE |
| P00006-01 | TRUE | FALSE | FALSE | FALSE |
| P00008-01 | FALSE | TRUE | FALSE | FALSE |
| P0000D-01 | TRUE | FALSE | FALSE | FALSE |
| P0000B-01 | TRUE | FALSE | FALSE | FALSE |
| P0000C-01 | TRUE | FALSE | FALSE | FALSE |
| P0000A-01 | FALSE | TRUE | FALSE | FALSE |
| P0000F-01 | FALSE | TRUE | FALSE | FALSE |
| P0000E-01 | TRUE | FALSE | FALSE | FALSE |
| P00002-01 | TRUE | FALSE | FALSE | FALSE |
| P00003-01 | FALSE | FALSE | TRUE | FALSE |
| P0000P-01 | TRUE | FALSE | FALSE | FALSE |
| P0000O-01 | FALSE | TRUE | FALSE | FALSE |
| P0000Q-01 | FALSE | FALSE | TRUE | FALSE |
| P0000R-01 | FALSE | FALSE | FALSE | TRUE |
| P0000S-01 | TRUE | FALSE | FALSE | FALSE |
| P0000U-01 | TRUE | FALSE | FALSE | FALSE |
| P00004-02 | TRUE | FALSE | FALSE | FALSE |
| P0000W-01 | FALSE | FALSE | FALSE | TRUE |
| P0000X-01 | TRUE | FALSE | FALSE | FALSE |
| P0000V-01 | TRUE | FALSE | FALSE | FALSE |
| P0000Y-01 | TRUE | FALSE | FALSE | FALSE |
| P0000Z-01 | TRUE | FALSE | FALSE | FALSE |
| P0000B-02 | TRUE | FALSE | FALSE | FALSE |
| P0000C-02 | TRUE | FALSE | FALSE | FALSE |
| P0000M-02 | FALSE | FALSE | TRUE | FALSE |
| P00012-01 | FALSE | FALSE | FALSE | TRUE |
| P000ll-01 | FALSE | TRUE | FALSE | FALSE |
| P000l0-01 | FALSE | FALSE | FALSE | TRUE |
| P0001V-02 | TRUE | FALSE | FALSE | FALSE |
| P00013-01 | FALSE | FALSE | FALSE | TRUE |
| P000lV-03 | TRUE | FALSE | FALSE | FALSE |
| P00016-02 | FALSE | TRUE | FALSE | FALSE |
| P000lA-01 | TRUE | FALSE | FALSE | FALSE |
| P000l7-01 | TRUE | FALSE | FALSE | FALSE |
| P00019-01 | FALSE | TRUE | FALSE | FALSE |
| P00015-01 | FALSE | TRUE | FALSE | FALSE |
| P00018-01 | TRUE | FALSE | FALSE | FALSE |
| P00014-01 | FALSE | FALSE | FALSE | TRUE |
| P000lC-01 | FALSE | TRUE | FALSE | FALSE |
| P000lB-01 | TRUE | FALSE | FALSE | FALSE |
| P000lD-01 | FALSE | TRUE | FALSE | FALSE |
| P000lV-01 | TRUE | FALSE | FALSE | FALSE |
| P0000H-01 | FALSE | TRUE | FALSE | FALSE |
| P00009-02 | TRUE | FALSE | FALSE | FALSE |
| P0000G-01 | TRUE | FALSE | FALSE | FALSE |
| P000lV-06 | TRUE | FALSE | FALSE | FALSE |
| P0000K-01 | FALSE | FALSE | TRUE | FALSE |
| P000OI-01 | TRUE | FALSE | FALSE | FALSE |
| PO000J-01 | FALSE | TRUE | FALSE | FALSE |
| P0000L-01 | TRUE | FALSE | FALSE | FALSE |
| P0000M-01 | TRUE | FALSE | FALSE | FALSE |
| P0000N-01 | TRUE | FALSE | FALSE | FALSE |
| P000IJ-01 | TRUE | FALSE | FALSE | FALSE |
| P00005-02 | FALSE | TRUE | FALSE | FALSE |
| P0000O-02 | FALSE | FALSE | TRUE | FALSE |
| P000lG-01 | TRUE | FALSE | FALSE | FALSE |
| P000lH-01 | TRUE | FALSE | FALSE | FALSE |
| P000II-01 | TRUE | FALSE | FALSE | FALSE |
| P000lV-13 | TRUE | FALSE | FALSE | FALSE |
| P000lM-02 | FALSE | TRUE | FALSE | FALSE |
| P000lP-01 | FALSE | TRUE | FALSE | FALSE |
| P000lQ-01 | TRUE | FALSE | FALSE | FALSE |
| P000lV-08 | TRUE | FALSE | FALSE | FALSE |
| P000lS-01 | TRUE | FALSE | FALSE | FALSE |
| P000lL-03 | FALSE | TRUE | FALSE | FALSE |
| P000lR-01 | TRUE | FALSE | FALSE | FALSE |
| P000lO-02 | TRUE | FALSE | FALSE | FALSE |
| P000lU-01 | TRUE | FALSE | FALSE | FALSE |
| P000lZ-01 | TRUE | FALSE | FALSE | FALSE |
| P0000T-02 | TRUE | FALSE | FALSE | FALSE |
| P000lT-01 | FALSE | FALSE | TRUE | FALSE |
| P0001X-01 | FALSE | FALSE | TRUE | FALSE |
| P000lY-01 | TRUE | FALSE | FALSE | FALSE |
| P0000Z-02 | FALSE | TRUE | FALSE | FALSE |
| P000lW-01 | FALSE | TRUE | FALSE | FALSE |
| P000lV-09 | TRUE | FALSE | FALSE | FALSE |
| P00020-01 | FALSE | TRUE | FALSE | FALSE |
| P000lN-01 | TRUE | FALSE | FALSE | FALSE |
| P000lO-01 | FALSE | FALSE | FALSE | TRUE |
| P0001V-12 | TRUE | FALSE | FALSE | FALSE |
| P00022-01 | TRUE | FALSE | FALSE | FALSE |
| P00024-01 | FALSE | TRUE | FALSE | FALSE |
| P00025-01 | TRUE | FALSE | FALSE | FALSE |
| P00028-01 | TRUE | FALSE | FALSE | FALSE |
| P00026-01 | FALSE | TRUE | FALSE | FALSE |
| P00027-01 | FALSE | TRUE | FALSE | FALSE |
| P0002D-01 | TRUE | FALSE | FALSE | FALSE |
| P0002G-01 | FALSE | TRUE | FALSE | FALSE |
| P0000Z-03 | TRUE | FALSE | FALSE | FALSE |
| P0002B-01 | FALSE | FALSE | FALSE | TRUE |
| P0002A-01 | TRUE | FALSE | FALSE | FALSE |
| P0002E-01 | FALSE | TRUE | FALSE | FALSE |
| P0002F-01 | FALSE | TRUE | FALSE | FALSE |
| P0002C-01 | FALSE | FALSE | FALSE | TRUE |
| P0000T-03 | TRUE | FALSE | FALSE | FALSE |
| P0002O-01 | TRUE | FALSE | FALSE | FALSE |
| P0000G-02 | TRUE | FALSE | FALSE | FALSE |
| P0002I-01 | TRUE | FALSE | FALSE | FALSE |
| P0002K-01 | FALSE | FALSE | FALSE | TRUE |
| P0002H-01 | FALSE | TRUE | FALSE | FALSE |
| P0002J-01 | FALSE | TRUE | FALSE | FALSE |
| P0002L-01 | FALSE | TRUE | FALSE | FALSE |
| P0002M-01 | TRUE | FALSE | FALSE | FALSE |
| P0000Z-04 | FALSE | TRUE | FALSE | FALSE |
| P0002N-01 | FALSE | TRUE | FALSE | FALSE |
| P0002R-01 | TRUE | FALSE | FALSE | FALSE |
| P0002P-01 | TRUE | FALSE | FALSE | FALSE |
| P0002G-02 | FALSE | TRUE | FALSE | FALSE |
| P0002S-01 | TRUE | FALSE | FALSE | FALSE |
| P0002T-01 | FALSE | FALSE | FALSE | TRUE |
| P0002U-01 | FALSE | TRUE | FALSE | FALSE |
| P0002V-01 | TRUE | FALSE | FALSE | FALSE |
| P00023-01 | TRUE | FALSE | FALSE | FALSE |
| P000lU-02 | FALSE | FALSE | TRUE | FALSE |
| P00033-01 | TRUE | FALSE | FALSE | FALSE |
| P00007-02 | TRUE | FALSE | FALSE | FALSE |
| P00029-02 | FALSE | FALSE | TRUE | FALSE |
| P00034-01 | TRUE | FALSE | FALSE | FALSE |
| P00035-01 | FALSE | TRUE | FALSE | FALSE |
| P0000B-03 | TRUE | FALSE | FALSE | FALSE |
| P00037-01 | FALSE | FALSE | TRUE | FALSE |
| P00036-01 | FALSE | TRUE | FALSE | FALSE |
| P00038-01 | FALSE | FALSE | TRUE | FALSE |
| P0003B-01 | TRUE | FALSE | FALSE | FALSE |
| P0003A-01 | FALSE | FALSE | TRUE | FALSE |
| P0003D-01 | TRUE | FALSE | FALSE | FALSE |
| P0003C-01 | TRUE | FALSE | FALSE | FALSE |
| P0003E-01 | FALSE | TRUE | FALSE | FALSE |
| P00039-02 | TRUE | FALSE | FALSE | FALSE |
| P0002X-02 | TRUE | FALSE | FALSE | FALSE |
| P0003F-01 | TRUE | FALSE | FALSE | FALSE |
| P00009-03 | FALSE | FALSE | TRUE | FALSE |
| P0002Z-01 | FALSE | FALSE | TRUE | FALSE |
| P0002W-01 | FALSE | FALSE | TRUE | FALSE |
| P0003I-01 | FALSE | FALSE | FALSE | TRUE |
| P0003H-01 | TRUE | FALSE | FALSE | FALSE |
| P000lZ-02 | TRUE | FALSE | FALSE | FALSE |
| P0002Y-01 | TRUE | FALSE | FALSE | FALSE |
| P00030-01 | FALSE | TRUE | FALSE | FALSE |
| P000lJ-02 | TRUE | FALSE | FALSE | FALSE |
| P0003N-01 | TRUE | FALSE | FALSE | FALSE |
| P0003J-01 | FALSE | TRUE | FALSE | FALSE |
| P0003G-02 | TRUE | FALSE | FALSE | FALSE |
| P0003K-01 | FALSE | TRUE | FALSE | FALSE |
| P0003M-01 | TRUE | FALSE | FALSE | FALSE |
| P0002Q-02 | FALSE | FALSE | TRUE | FALSE |
| P00032-01 | FALSE | TRUE | FALSE | FALSE |
| P0003l-01 | FALSE | TRUE | FALSE | FALSE |
| P0006H-01 | FALSE | TRUE | FALSE | FALSE |
| P0006F-01 | FALSE | TRUE | FALSE | FALSE |
| P00057-02 | TRUE | FALSE | FALSE | FALSE |
| P0006I-01 | FALSE | TRUE | FALSE | FALSE |
| P0006K-01 | TRUE | FALSE | FALSE | FALSE |
| P0006E-01 | TRUE | FALSE | FALSE | FALSE |
| P0006G-01 | FALSE | FALSE | FALSE | TRUE |
| P0006L-01 | FALSE | FALSE | FALSE | TRUE |
| P0002V-03 | TRUE | FALSE | FALSE | FALSE |
| P0006M-01 | TRUE | FALSE | FALSE | FALSE |
| P0006N-01 | FALSE | TRUE | FALSE | FALSE |
| P0006O-01 | TRUE | FALSE | FALSE | FALSE |
| P0002B-03 | TRUE | FALSE | FALSE | FALSE |
| P0006R-01 | TRUE | FALSE | FALSE | FALSE |
| P0006X-01 | FALSE | TRUE | FALSE | FALSE |
| P0006Q-01 | TRUE | FALSE | FALSE | FALSE |
| P0006S-01 | FALSE | FALSE | TRUE | FALSE |
| P0006W-01 | FALSE | TRUE | FALSE | FALSE |
| P0006Y-01 | FALSE | FALSE | TRUE | FALSE |
| P0006T-01 | FALSE | FALSE | FALSE | TRUE |
| P0006V-01 | FALSE | TRUE | FALSE | FALSE |
| P0007B-01 | TRUE | FALSE | FALSE | FALSE |
| P0007A-01 | TRUE | FALSE | FALSE | FALSE |
| P0006C-02 | TRUE | FALSE | FALSE | FALSE |
| P0006Z-01 | TRUE | FALSE | FALSE | FALSE |
| P00072-01 | FALSE | FALSE | TRUE | FALSE |
| P0006P-02 | FALSE | TRUE | FALSE | FALSE |
| P0003P-01 | TRUE | FALSE | FALSE | FALSE |
| P00009-04 | FALSE | FALSE | TRUE | FALSE |
| P0000M-03 | TRUE | FALSE | FALSE | FALSE |
| P000l2-02 | FALSE | FALSE | TRUE | FALSE |
| P0003R-01 | FALSE | TRUE | FALSE | FALSE |
| P0003X-01 | FALSE | FALSE | FALSE | TRUE |
| P0003S-01 | FALSE | TRUE | FALSE | FALSE |
| P0003T-01 | FALSE | TRUE | FALSE | FALSE |
| P0003W-01 | FALSE | FALSE | TRUE | FALSE |
| P0003U-01 | TRUE | FALSE | FALSE | FALSE |
| P0003Y-01 | TRUE | FALSE | FALSE | FALSE |
| P0003V-01 | TRUE | FALSE | FALSE | FALSE |
| P0000C-03 | TRUE | FALSE | FALSE | FALSE |
| P0000H-02 | TRUE | FALSE | FALSE | FALSE |
| P0000O-03 | FALSE | TRUE | FALSE | FALSE |
| P00040-01 | TRUE | FALSE | FALSE | FALSE |
| P0003Z-01 | FALSE | FALSE | TRUE | FALSE |
| P0003N-02 | TRUE | FALSE | FALSE | FALSE |
| P00042-01 | TRUE | FALSE | FALSE | FALSE |
| P00041-01 | TRUE | FALSE | FALSE | FALSE |
| P0000J-02 | FALSE | TRUE | FALSE | FALSE |
| P00043-01 | TRUE | FALSE | FALSE | FALSE |
| P00044-01 | TRUE | FALSE | FALSE | FALSE |
| P0000L-02 | FALSE | TRUE | FALSE | FALSE |
| P000ll-02 | FALSE | TRUE | FALSE | FALSE |
| P0003Q-02 | TRUE | FALSE | FALSE | FALSE |
| P0002M-02 | TRUE | FALSE | FALSE | FALSE |
| P00049-01 | TRUE | FALSE | FALSE | FALSE |
| P00070-01 | TRUE | FALSE | FALSE | FALSE |
| P0005J-02 | TRUE | FALSE | FALSE | FALSE |
| P0006J-02 | FALSE | FALSE | FALSE | TRUE |
| P0006U-02 | FALSE | TRUE | FALSE | FALSE |
| P00073-01 | FALSE | FALSE | TRUE | FALSE |
| P00075-01 | FALSE | FALSE | TRUE | FALSE |
| P00078-01 | FALSE | TRUE | FALSE | FALSE |
| P0003Z-03 | FALSE | TRUE | FALSE | FALSE |
| P0002L-03 | FALSE | FALSE | FALSE | TRUE |
| P00057-03 | TRUE | FALSE | FALSE | FALSE |
| P00076-01 | FALSE | TRUE | FALSE | FALSE |
| P00077-01 | TRUE | FALSE | FALSE | FALSE |
| P0002M-04 | FALSE | FALSE | FALSE | TRUE |
| P0007C-01 | FALSE | FALSE | FALSE | TRUE |
| P00079-01 | FALSE | FALSE | FALSE | TRUE |
| P0007D-01 | FALSE | TRUE | FALSE | FALSE |
| P0002F-02 | TRUE | FALSE | FALSE | FALSE |
| P0007E-01 | FALSE | TRUE | FALSE | FALSE |
| P0007F-01 | TRUE | FALSE | FALSE | FALSE |
| P0007G-01 | TRUE | FALSE | FALSE | FALSE |
| P0002A-03 | TRUE | FALSE | FALSE | FALSE |
| P0005U-03 | TRUE | FALSE | FALSE | FALSE |
| P0007H-01 | TRUE | FALSE | FALSE | FALSE |
| P0007l-01 | TRUE | FALSE | FALSE | FALSE |
| P00068-02 | FALSE | TRUE | FALSE | FALSE |
| P0006D-01 | TRUE | FALSE | FALSE | FALSE |
| P0005W-02 | FALSE | TRUE | FALSE | FALSE |
| P0003M-02 | FALSE | FALSE | TRUE | FALSE |
| P0004Y-02 | FALSE | FALSE | FALSE | TRUE |
| P0004X-01 | TRUE | FALSE | FALSE | FALSE |
| P00056-01 | TRUE | FALSE | FALSE | FALSE |
| P00057-01 | TRUE | FALSE | FALSE | FALSE |
| P00058-01 | TRUE | FALSE | FALSE | FALSE |
| P0002T-02 | TRUE | FALSE | FALSE | FALSE |
| P00059-01 | FALSE | TRUE | FALSE | FALSE |
| P0000P-02 | TRUE | FALSE | FALSE | FALSE |
| P0005A-01 | TRUE | FALSE | FALSE | FALSE |
| P0005B-01 | FALSE | FALSE | FALSE | TRUE |
| P0000J-03 | FALSE | TRUE | FALSE | FALSE |
| P0005C-01 | TRUE | FALSE | FALSE | FALSE |
| P0002I-02 | TRUE | FALSE | FALSE | FALSE |
| P0003O-02 | FALSE | FALSE | FALSE | TRUE |
| P0000A-03 | FALSE | TRUE | FALSE | FALSE |
| P0004Y-03 | FALSE | TRUE | FALSE | FALSE |
| P000lU-04 | TRUE | FALSE | FALSE | FALSE |
| P000lK-02 | TRUE | FALSE | FALSE | FALSE |
| P0002H-02 | FALSE | TRUE | FALSE | FALSE |
| P00027-02 | TRUE | FALSE | FALSE | FALSE |
| P0002D-02 | TRUE | FALSE | FALSE | FALSE |

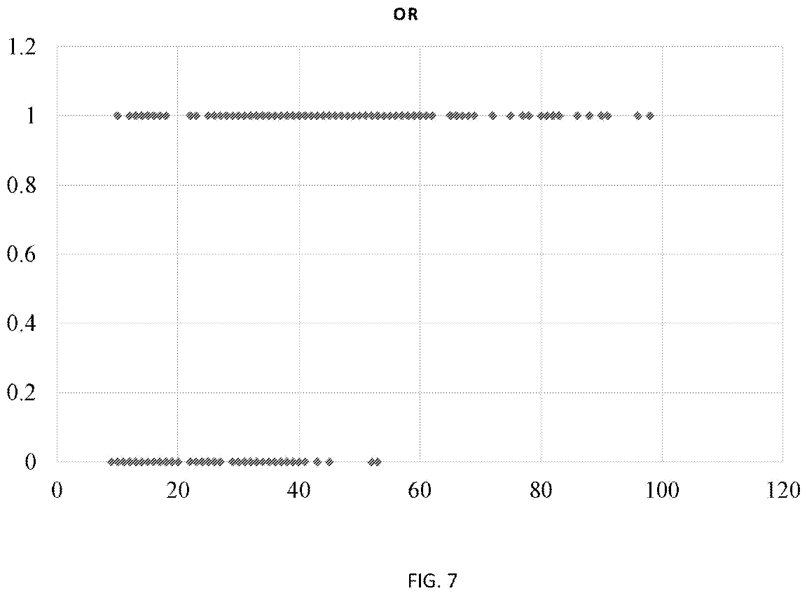

TABLE 3B provides a more succinct tabulation of the individual results presented in TABLE 3A. FIG. 7 is a plot of OR (m-cIDNA 25, total protein 25) vs. Q-Score for each sample.

TABLE 3B

| | Column Labels | | |
|---|---|---|---|
| Count of OR | FALSE | TRUE | Grand Total |
| OK | 87 | 36 | 123 |
| REJECT | 30 | 176 | 206 |
| Grand Total | 117 | 212 | 329 |

The urine dipstick test is a screening assay, which could detect positive cases (true disease). Because the test readout could be altered depending on the test features (threshold of detection and threshold of quantification), alkalinity of the urine, and presence of infections, We have utilized the combination of two separate markers to provide a screening test for detection of positive cases.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein.

Numerous variations may be made by persons skilled in the art without departure from the spirit of the invention. The scope of the invention will be measured by the appended claims and their equivalents. The abstract and the title are snot to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, i]6.

1. A lateral flow device for detecting the presence or absence of methylated cIDNA and one or more protein(s) in a biological sample comprising:
a sample application area;
a capture area comprising a membrane having at least:
a first detectable moiety attached thereto whereby the first detectable moiety specifically binds to a methylated cell free nucleic acid (m-cIDNA) to form a detectable complex;
a second detectable moiety attached thereto whereby the second detectable moiety specifically reacts with one or more protein(s); and
a flow path from the sample application area to the capture area.

2. The device of claim 1, wherein the first detectable moiety has a threshold of detection for forming a detectable m-cIDNA complex of at least 15 ng/ml.

3. The device of claim 2, wherein the first detectable moiety has a threshold of detection for forming a detectable m-cIDNA complex of at least 20 ng/mL.

4. The device of claim 3, wherein the first detectable moiety has a threshold of detection for forming a detectable m-cIDNA complex of at least 25 ng/mL.

5. The device of claim 1, wherein the second detectable moiety has a threshold of detection for specifically reacting with one or more protein(s) of at least 15 ng/mL.

6. The device of claim 5, wherein the second detectable moiety has a threshold of detection for specifically reacting with one or more protein(s) of at least 20 ng/mL.

7. The device of claim 6, wherein the second detectable moiety has a threshold of detection for specifically reacting with one or more protein(s) of at least 25 ng/mL.

8. (canceled)

9. The device of any one of claim 1, wherein a combined threshold of detection for forming a detectable m-cIDNA complex of at least 25 ng/ml and for forming a detectable m-cIDNA complex of at least 25 ng/ml identifies an allograft rejection with at least 65% specificity.

10. The device of claim 1, wherein the second detectable moiety that specifically reacts with one or more protein(s) substantially reacts with albumin.

11. (canceled)

12. The device of claim 1, wherein the first detectable moiety is an antibody.

13. The device of claim 1, wherein the second detectable moiety is a color changing dye.

14. The device of claim 13, wherein the color changing dye preferably bind albumin.

15. The device of claim 1, wherein the capture area further comprises a control detectable moiety.

16. The device of claim 15, wherein the control detectable moiety is a species specific IgG.

17. The device of claim 16, wherein the species is human.

18. The device of claim 17, wherein the species is dog.

19. A method for using a lateral flow device of claim 1 in detecting a kidney allograft rejection.

20. A kit comprising a lateral flow device of claim 1 and instructions for use therefore.

21. A lateral flow device for detecting the presence or absence of methylated cIDNA and one or more protein(s), said device comprising a test strip having a first and second end and comprising:
(a) a sample receiving zone at or adjacent said first end of said test strip for receiving an aliquot of a bodily fluid sample;
(b) a first capture zone in lateral flow contact with said sample receiving zone, said capture zone comprising at least a first detectable moiety attached thereto and coupled to a first binding partner which specifically binds to a methylated cell free nucleic acid to form a detectable complex;
(c) a second capture zone in lateral flow contact with said first labeling zone, said capture zone comprising a dye attached thereto which specifically binds to one or more protein(s); and
(c) optionally a third capture zone in lateral flow contact with said second capture zone, said capture zone comprising a control detectable moiety attached thereto which binds to a control; and
(d) optionally an absorbent zone positioned at or adjacent said second end of said test strip in lateral flow contact with capture zones.

22. The device of claim 21, wherein the first detectable moiety has a threshold of detection for forming a detectable m-cIDNA complex of at least 15 ng/ml.

23. (canceled)

24. The device of claim 21, wherein the first detectable moiety has a threshold of detection for forming a detectable m-cIDNA complex of at least 25 ng/ml.

25. (canceled)

26. (canceled)

27. (canceled)

28. (canceled)

29. The device of any one of claim 21, wherein a combined threshold of detection for forming a detectable m-cIDNA complex of at least 25 ng/ml and for forming a detectable m-cIDNA complex of at least 25 ng/ml identifies an allograft rejection with at least 65% specificity.

30. The device of claim 21, wherein the dye preferentially reacts with albumin.

31. (canceled)

32. The device of claim 21, wherein the first detectable moiety is an antibody.

33. The device of claim 21, wherein the dye is a color changing dye.

34. The device of claim 33, wherein the color changing dye preferably binds albumin.

35. The device of claim 21, wherein the capture area further comprises a fourth capture zone for detection of an additional moiety.

36. The device of claim 15, wherein the control detectable moiety is a species specific IgG.

37. The device of claim 36, wherein the species is human.

38. The device of claim 37, wherein the species is dog.

39. A method for using a lateral flow device of claim 21 in detecting a kidney allograft rejection.

40. A kit comprising a lateral flow device of claim 21 and instructions for use therefore.
